SEQUENCE LISTING (120 sequences)

SEQ ID NO: 1 - protein, 11 aa, synthetic construct
RASQSVNSNLA

SEQ ID NO: 2 - protein, 7 aa, synthetic construct
GASTRAT

SEQ ID NO: 3 - protein, 9 aa, synthetic construct
QQYNNWPFT

SEQ ID NO: 4 - protein, 7 aa, synthetic construct
RNSAVWN

SEQ ID NO: 5 - protein, 18 aa, synthetic construct
RTYYRSKWYNDYAVSVKS

SEQ ID NO: 6 - protein, 13 aa, synthetic construct
GLLQNNFYYYMDV

SEQ ID NO: 7 - protein, 125 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 8 - protein, 107 aa, synthetic construct
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIK

SEQ ID NO: 9 - protein, 252 aa, synthetic construct
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 10 - protein, 469 aa, synthetic construct
MLLLVTSLLLCELPHPAFLLIPEIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 11 aa, synthetic construct
RASQSVSSNLA

SEQ ID NO: 12 - protein, 7 aa, synthetic construct
GASTRAT

SEQ ID NO: 13 - protein, 9 aa, synthetic construct
QQYNNWPFT

SEQ ID NO: 14 - protein, 7 aa, synthetic construct
RNSAVWN

SEQ ID NO: 15 - protein, 18 aa, synthetic construct
RTYYRSKWYNDYAVSVKS

SEQ ID NO: 16 - protein, 13 aa, synthetic construct
GLLQNQFYYYMDV

SEQ ID NO: 17 - protein, 125 aa, synthetic construct
QVQLQQSGPGLVKPSQTLSLTCAISGDSVSRNSAVWNWIRQSPSRGLEWLGRTYYRSKWYNDYAVSVKSRITINPDTSKNQFSLQLNSVTPEDTAVYYCARGLLQNQFYYYMDVWGKGTTVTVSS

SEQ ID NO: 18 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 19-20 - skipped (2 entries)

SEQ ID NO: 21 - protein, 16 aa, synthetic construct
RSSQSVVHSNGNTYLE

SEQ ID NO: 22 - protein, 7 aa, synthetic construct
KVSNRFS

SEQ ID NO: 23 - protein, 9 aa, synthetic construct
FQGSHVPYT

SEQ ID NO: 24 - protein, 5 aa, synthetic construct
SYGMS

SEQ ID NO: 25 - protein, 17 aa, synthetic construct
TISSGGSYTFYPDIMKG

SEQ ID NO: 26 - protein, 13 aa, synthetic construct
RGYGTIYTFSFDS

SEQ ID NO: 27 - protein, 122 aa, synthetic construct
EVQLVESGGDLVKPGGSLKLSCAASGFSFSSYGMSWVRQTPDKRLEWVATISSGGSYTFYPDIMKGRFTISRDNAMNTLYLQMSSLKSEDSAMYYCARRGYGTIYTFSFDSWGQGTTLTVSS

SEQ ID NO: 28 - protein, 112 aa, synthetic construct
DVLMTQTPLSLPVSLGDQASISCRSSQSVVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 29-30 - skipped (2 entries)

SEQ ID NO: 31 - protein, 16 aa, synthetic construct
RSSQSVVHSNANTYLE

SEQ ID NO: 32 - protein, 7 aa, synthetic construct
KVSNRFS

SEQ ID NO: 33 - protein, 9 aa, synthetic construct
FQGSHVPYT

SEQ ID NO: 34 - protein, 5 aa, synthetic construct
SYGMS

SEQ ID NO: 35 - protein, 17 aa, synthetic construct
TISSGGSYTFYPDIMKG

SEQ ID NO: 36 - protein, 13 aa, synthetic construct
RGYGTIYTFSFDA

SEQ ID NO: 37 - protein, 122 aa, synthetic construct
EVQLVESGGGLVKPGGSLRLSCAASGFTFSSYGMSWVRQAPGKRLEWVATISSGGSYTFYPDIMKGRFTISRDNSKNTLYLQMNSLRAEDTAVYYCARRGYGTIYTFSFDAWGQGTTLTVSS

SEQ ID NO: 38 - protein, 112 aa, synthetic construct
DVVMTQSPLSLPVTLGQPASISCRSSQSVVHSNANTYLEWYLQKPGQSPQLLIYKVSNRFSGVPDRFSGSGSGTDFTLKISRVEAEDVGVYYCFQGSHVPYTFGQGTKLEIK

SEQ ID NO: 39-40 - skipped (2 entries)

SEQ ID NO: 41 - protein, 16 aa, synthetic construct
RSSQSVVHSNANTYLE

SEQ ID NO: 42 - protein, 7 aa, synthetic construct
KVSNRFS

SEQ ID NO: 43 - protein, 9 aa, synthetic construct
FQGSHVPYT

SEQ ID NO: 44 - protein, 5 aa, synthetic construct
SYGMS

SEQ ID NO: 45 - protein, 17 aa, synthetic construct
TISSGGSYTFYPDIMKG

SEQ ID NO: 46 - protein, 13 aa, synthetic construct
RGYGTIYTFSFDA

SEQ ID NO: 47 - protein, 122 aa, synthetic construct
EVQLLESGGGLVQPGGSLRLSCAASGFTFSSYGMSWVRQAPGKRLEWVSTISSGGSYTFYPDIMKGRFTISRDNSKNTLYLQMNSLRAEDTAVYYCARRGYGTIYTFSFDAWGQGTTLTVSS

SEQ ID NO: 48 - protein, 112 aa, synthetic construct; identical to SEQ ID NO: 38

SEQ ID NO: 49-56 - skipped (8 entries)

SEQ ID NO: 57 - protein, 120 aa, synthetic construct
EVQLVESGGGLVQPGGSLRLSCAASGFTFSSFAMTWVRQAPGKGLEWVSVITYSGGRTYYADSVKGRFTISRDNSKNTLYLQMNSLRAEDTAVYFCAKDRIAAVGPFDYWGQGTLVTVSS

SEQ ID NO: 58 - protein, 107 aa, synthetic construct
DIQLTQSPSFLSASVGDRVTITCRASQGISVYLAWYQQEPGKAPKLLIYAASTLQSGVPSRFSGSGSGTEFTLTISSLQPEDFATYYCQQLNSYPRTFGQGTKVEIK

SEQ ID NO: 59-66 - skipped (8 entries)

SEQ ID NO: 67 - protein, 116 aa, synthetic construct
QVQLVQSGAEVKKPGASVKVSCKASGYTFTSYGISWVRQAPGQGLEWMGWISGYTGNTNYAQKLQGRVTMTADTSTSTAYMELRSLRSDDTAVYYCARGGSYFDYWGQGTLVTVSS

SEQ ID NO: 68 - protein, 108 aa, synthetic construct
DIQMTQSPSTLSASVGDRVTITCRASQSISRWLAWYQQKPGKAPKLLIYKASSLESGVPSRFSGSGSGTEFTLTISSLQPDDFATYYCQQFNSFSPITFGQGTRLEIK

SEQ ID NO: 69-76 - skipped (8 entries)

SEQ ID NO: 77 - protein, 117 aa, synthetic construct
QVQLQQPGAELVKPGASVKLSCKASGYTFTSYWMEWVKQRPGQGLEWIGMIHPNSGITNYNERFKNKATLTVDKSSSTAYMQLSSLTSEDSAVYYCARDHYYILAYWGQGTLVTVSA

SEQ ID NO: 78 - protein, 112 aa, synthetic construct; identical to SEQ ID NO: 28

SEQ ID NO: 79-86 - skipped (8 entries)

SEQ ID NO: 87 - protein, 120 aa, synthetic construct
QVQLQQPGADLVKPGASVKMSCKASGHTFTNYWVTWVKQRPGQGLEWIGNFYPGSGIIKYNENFRSKATLTVDISSSTAYMQLSSLTSEDSAVYYCARSKLGDSFYFDYWGQGTTLTVSS

SEQ ID NO: 88 - protein, 112 aa, synthetic construct
DVVMTQTPLSLPVSLGNQASISCRSSQSLVHSNGNTYLHWYLQKPGQSPKLLIYKVSNRFSGVPDRFSGSGSGTDFTLKISRVEAEDLGVYFCSQSTHVPLTFGAGTKLEIK

SEQ ID NO: 89-100 - skipped (12 entries)

SEQ ID NO: 101 - DNA, 756 bp, synthetic construct
  source 1..756: mol_type = other DNA
gagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctcc

SEQ ID NO: 102 - DNA, 1410 bp, synthetic construct
  source 1..1410: mol_type = other DNA
atgctgctccttgtcacatcactgctgctctgcgaactgccccaccctgcattcctcctgatccccgagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctccggctccgaatcaaaatacggtccgccatgcccaccgtgccccttctgggtgctcgtggtcgtcggaggggttctggcctgctactccctgctggtcaccgtggcgtttatcatcttctgggtgaagcggggaaggaagaagctactgtacattttcaagcagcctttcatgcggcctgtgcagaccacccaggaagaggacggctgttcctgccggttccccgaggaagaggaagggggttgcgagctgcgcgtgaagttcagcaggagcgctgatgccccagcgtaccaacaggggcaaaaccagttgtacaacgaactgaaccttggtcggcgcgaagagtacgacgtgcttgacaagcgccgcggcagagatcccgagatgggtggaaagccgcggcggaagaatccgcaggaagggctctacaacgagctccagaaggacaagatggccgaagcctacagcgaaatcgggatgaagggcgaaagacgccggggaaaaggacacgacggactgtaccaggggttgtcgaccgcgaccaaggacacctacgacgccctgcatatgcaagccttgccgccgagatga

SEQ ID NO: 103 - DNA, 762 bp, synthetic construct
  source 1..762: mol_type = other DNA
gatgttttgatgacccaaactcctctctccctgcctgtcagtcttggagatcaagcctccatctcttgcagatctagtcagagtgttgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaaggaggcggaggatctggtggtggtggatctggcggcggaggaagtggtggcggaggctctgaggtgcagctggtggagtctgggggagacttagtgaagcctggagggtccctgaaactctcctgtgccgcctctggattctctttctcctcttatggcatgtcttgggttcgccagactccagacaagaggctggaatgggtcgcaaccattagtagtggtggtagttacaccttctatcccgacattatgaaggggcgattcaccatctccagagacaatgccatgaacaccctgtacctgcaaatgagcagtctgaagtctgaggactcagccatgtattactgtgcaagacggggctacggtactatctacacgttttcctttgactcctggggccaaggcaccactctcacagtctccagc

SEQ ID NO: 104 - DNA, 582 bp, synthetic construct
  source 1..582: mol_type = other DNA
atgggacgcgggctgcttcgaggactctggccacttcatatcgtgttgtggactcgcatcgcttcaaccattccgccgcacgtgcagaagtccgtgaacaatgacatgatcgtgaccgacaacaacggtgcagtgaagttcccacagctgtgcaagttctgcgatgtcagattcagcacttgcgacaaccagaagtcctgcatgtcaaactgctccatcacctccatctgcgagaagcctcaagaggtctgcgtggccgtgtggcggaagaacgacgagaacatcaccctggaaaccgtgtgccacgatccgaagctgccttatcacgacttcattctggaagatgccgcctcgcccaagtgtatcatgaaagaaaagaaaaagcccggagaaacgttcttcatgtgctcgtgtagctccgacgagtgcaacgacaacattatctttagcgaagagtacaacacttccaaccctgacctcctgctcgtgatttttcaagtcaccggcatttccctgctgcccccgctgggcgtggcgatctcggtgatcattatcttctactgttaccgggtcaataggcag

SEQ ID NO: 105 - protein, 194 aa, synthetic construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 106 - protein, 684 aa, synthetic construct
MLLLVTSLLLCELPHPAFLLIPEIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 107 - DNA, 2052 bp, synthetic construct
  source 1..2052: mol_type = other DNA
atgctgctccttgtcacatcactgctgctctgcgaactgccccaccctgcattcctcctgatccccgagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctccggctccgaatcaaaatacggtccgccatgcccaccgtgccccttctgggtgctcgtggtcgtcggaggggttctggcctgctactccctgctggtcaccgtggcgtttatcatcttctgggtgaagcggggaaggaagaagctactgtacattttcaagcagcctttcatgcggcctgtgcagaccacccaggaagaggacggctgttcctgccggttccccgaggaagaggaagggggttgcgagctgcgcgtgaagttcagcaggagcgctgatgccccagcgtaccaacaggggcaaaaccagttgtacaacgaactgaaccttggtcggcgcgaagagtacgacgtgcttgacaagcgccgcggcagagatcccgagatgggtggaaagccgcggcggaagaatccgcaggaagggctctacaacgagctccagaaggacaagatggccgaagcctacagcgaaatcgggatgaagggcgaaagacgccggggaaaaggacacgacggactgtaccaggggttgtcgaccgcgaccaaggacacctacgacgccctgcatatgcaagccttgccgccgagaggatccggagaggggaggggaagcctcctcacttgcggcgatgtggaggaaaacccgggtcctatgggacgcgggctgcttcgaggactctggccacttcatatcgtgttgtggactcgcatcgcttcaaccattccgccgcacgtgcagaagtccgtgaacaatgacatgatcgtgaccgacaacaacggtgcagtgaagttcccacagctgtgcaagttctgcgatgtcagattcagcacttgcgacaaccagaagtcctgcatgtcaaactgctccatcacctccatctgcgagaagcctcaagaggtctgcgtggccgtgtggcggaagaacgacgagaacatcaccctggaaaccgtgtgccacgatccgaagctgccttatcacgacttcattctggaagatgccgcctcgcccaagtgtatcatgaaagaaaagaaaaagcccggagaaacgttcttcatgtgctcgtgtagctccgacgagtgcaacgacaacattatctttagcgaagagtacaacacttccaaccctgacctcctgctcgtgatttttcaagtcaccggcatttccctgctgcccccgctgggcgtggcgatctcggtgatcattatcttctactgttaccgggtcaataggcag

SEQ ID NO: 108 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKRYFDYWGQGTMVTVSS

SEQ ID NO: 109 - protein, 110 aa, synthetic construct
SYELTQPPSASGTPGQRVTISCSGGSSNIGSNTVNWFRQLPGTAPKLLVYFNNQRPSGVPDRFSGSKSGTSASLAIGGLQSDDEADYYCVAWDDSLNAPVFGGGTKVTVL

SEQ ID NO: 110 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSS

SEQ ID NO: 111 - protein, 110 aa, synthetic construct
QSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVL

SEQ ID NO: 112 - protein, 10 aa, synthetic construct
GFTFSSYAMS

SEQ ID NO: 113 - protein, 17 aa, synthetic construct
AISGSGGSTYYADSVKG

SEQ ID NO: 114 - protein, 7 aa, synthetic construct
GKRYFDY

SEQ ID NO: 115 - protein, 13 aa, synthetic construct
SGGSSNIGSNTVN

SEQ ID NO: 116 - protein, 7 aa, synthetic construct
FNNQRPS

SEQ ID NO: 117 - protein, 11 aa, synthetic construct
VAWDDSLNAPV

SEQ ID NO: 118 - protein, 13 aa, synthetic construct
SGGSSDIGSNTVN

SEQ ID NO: 119 - protein, 7 aa, synthetic construct
YNNQRPS

SEQ ID NO: 120 - protein, 11 aa, synthetic construct
ATWDDRMYSPV

(19) United States
(12) Patent Application Publication
(10) Pub. No.: US 2024/0117309 A1
(43) Pub. Date: Apr. 11, 2024

(54) METHODS FOR EXPANDING T CELL POPULATIONS

(71) Applicant: MedImmune, LLC, Gaithersburg, MD (US)

(72) Inventors: Jinlin JIANG, Gaithersburg, MD (US); Emily BOSCO, Gaithersburg, MD (US); Christina GRIGORIADOU, Gaithersburg, MD (US); Maria Letizia GIARDINO TORCHIA, Cambridge (GB); Gordon MOODY, Gaithersburg (GB); Attilio BONDANZA, Cambridge (GB); Nina CHU, Gaithersburg, MD (US)

(21) Appl. No.: 18/351,821

(22) Filed: Jul. 13, 2023

Related U.S. Application Data
(60) Provisional application No. 63/368,550, filed on Jul. 15, 2022.

(51) Int. Cl. C12N 5/0783; C07K 16/30; C07K 16/40

(52) U.S. Cl. CPC C12N 5/0636; C07K 16/303; C07K 16/40; C12N 2501/2302; C12N 2501/2321; C12N 2510/00

(57) ABSTRACT

Provided herein are methods for manufacturing, expanding, and/or generating genetically modified T cells comprising a chimeric antigen receptor (CAR) or T-cell receptor (TCR).

### CROSS-REFERENCE TO RELATED APPLICATION

This application claims the priority benefit of U.S. Provisional Application No. 63/368,550 filed Jul. 15, 2022, which is incorporated herein by reference in its entirety.

### REFERENCE TO SEQUENCE LISTING SUBMITTED ELECTRONICALLY

The content of the electronically submitted sequence listing (CARTSTEAP2-300-US-NP.xml; Size: 81,161 bytes; and Date of Creation: Jul. 10, 2023) submitted in this application is incorporated herein by reference in its entirety.

### BACKGROUND OF THE DISCLOSURE

The generation of tumor-specific T lymphocytes by genetic modification to express chimeric antigen receptors (CARs) is gaining traction as a form of synthetic biology eliciting powerful antitumor effects (Jena et al., 2010, Blood. 116:1035-1044; Bonini et al., 2011, Biol Blood Marrow Transplant 17(1 Suppl):S15-20; Restifo et al., 2012, Nat Rev Immunol 12:269-281; Kohn et al., 2011, Mol Ther 19:432-438; Savoldo et al., 2011, J Clin Invest 121:1822-1825; Ertl et al., 2011, Cancer Res 71:3175-3181). Because the specificity is conferred by antibody fragments, the CAR-T cells are not MHC restricted and are therefore more practical than approaches based on T cell receptors that require MHC matching.

As such, CAR-T cell therapy represents a major advancement in personalized cancer treatment. In this strategy, a patient's own T cells are genetically engineered to express a synthetic receptor that binds a tumor antigen. CAR-T cells are then expanded for clinical use and infused back into the patient's body to attack and destroy chemotherapy-resistant cancer. Dramatic clinical responses and high rates of complete remission have been observed in the setting of CAR-T cell therapy of B-cell malignancies. This resulted in two recent FDA approvals of CAR-T cells directed against the CD19 protein for treatment of acute lymphoblastic leukemia and diffuse large B-cell lymphoma. Thus, CAR-T cells are arguably one of the first successful examples of synthetic biology and personalized cellular cancer therapy to become commercially available.

Despite the recent successes of CAR-T cell therapy, there is still a need in the art to better improve T cell expansion methods.

### SUMMARY OF THE DISCLOSURE

The present disclosure is related to a method of expanding a population of T cells comprising: (a) isolating CD3+ T cells from a sample; (b) culturing the CD3+ T cells in a culture media that comprises human interleukin 21 (IL-21); (c) activating the CD3+ T cells; (d) transducing the CD3+ T cells with a vector comprising a nucleic acid encoding a chimeric antigen receptor (CAR) or a T-Cell Receptor (TCR) to produce CAR-T cells or T-cell Receptor (TCR) cells; (e) culturing the CAR-T cells in a medium; and (f) harvesting the CAR-T cells or T-cell Receptor (TCR) cells. The present disclosure is also related to a method of manufacturing a T cell therapeutic comprising: (a) obtaining a sample comprising a population of CD3+ T cells; (b) culturing the CD3+ T cells in a culture media that comprises human interleukin 21 (IL-21); (c) activating the CD3+ T cells; (d) transducing the CD3+ T cells with a vector comprising a nucleic acid encoding a chimeric antigen receptor (CAR) or a T-Cell Receptor (TCR) to produce CAR-T cells or T-cell Receptor (TCR) cells; (e) culturing the CAR-T cells or T-cell Receptor (TCR) cells in a medium; and (f) harvesting the CAR-T cells or T-cell Receptor (TCR) cells. The present disclosure is also related to a method of expanding a population of T cells comprising: (a) isolating CD4+ and CD8+ T cells from a sample to form a population of CD3+ T cells; (b) culturing the CD3+ T cells in a culture media containing human interleukin 21 (IL-21); (c) activating the CD3+ T cells; (d) transducing the CD3+ T cells with a vector comprising a nucleic acid encoding a chimeric antigen receptor (CAR) or a T-Cell Receptor (TCR) to produce CAR-T cells or T-cell Receptor (TCR) cells; (e) culturing the CAR-T cells or T-cell Receptor (TCR) cells in a medium; and (f) harvesting the CAR-T cells or T-cell Receptor (TCR) cells. In some aspects, the culture media further comprises human interleukin 2 (IL-2).

In some aspects, part (d) comprises transducing the CD3+ T cells with a vector comprising a nucleic acid encoding a CAR to produce CAR-T cells. In some aspects, part (d) comprises transducing the CD3+ T cells with a vector comprising a nucleic acid encoding a TCR to produce TCR cells. In some aspects, about from 1×10⁶ to about 1×10⁹ CD3+ T cells are cultured in step (b) in the culture media. In some aspects, the sample is an enriched apheresis product collected via leukapheresis. In some aspects, the CD3+ T cells in step (c) are cultured for about one day or about two days. In some aspects, the CD3+ T cells in step (c) are activated with agonists of CD2, CD3, CD28, or any combination thereof. In some aspects, the CD3+ T cells in step (c) are activated with magnetic microbeads. In some aspects, the CD3+ T cells in step (c) are activated with an anti-CD3 antibody or CD3-binding fragment thereof, and an anti-CD28 antibody or a CD28-binding fragment thereof. In some aspects, the anti-CD3 antibody or CD3-binding fragment thereof, and the anti-CD28 antibody or a CD28-binding fragment thereof are coupled to a magnetic microbead. In some aspects, the CAR-T cells or TCR cells are cultured in step (e) from about two to about ten days. In some aspects, the CAR-T cells or TCR cells are cultured in step (e) from about four to about six days. In some aspects, the CAR-T T cells are cultured in step (e) for about four days. In some aspects, the CAR-T cells or TCR cells are cultured in step (e) for about six days. In some aspects, the concentration of human IL-21 is from about 0.01 U/mL to about 0.3 U/mL, and the concentration of human IL-2 is from about 5 IU/mL to about 100 IU/mL. In some aspects, the concentration of human IL-21 is about 0.19 U/mL. In some aspects, the concentration of human IL-2 is about 40 IU/mL. In some aspects, the CD3+ T cells are agitated during step (b). The methods of the present disclosure are related to a method of manufacturing a T cell therapeutic comprising: (a) isolating CD4+ and CD8+ T cells from a sample to form a population of CD3+ T cells; (b) culturing the CD3+ T cells in a culture media that comprises human interleukin 2 at a concentration of 40 IU/mL and human interleukin 21 at a concentration of 0.19 U/mL; (c) activating the CD3+ T cells with a magnetic bead comprising an anti-CD3 antibody or CD3-binding fragment thereof, and an anti-CD28 antibody or a CD28-binding fragment thereof; (d) transducing the CD3+ T cells with a lentiviral vector virus comprising a nucleic acid encoding a chimeric antigen receptor (CAR) to produce CAR-T cells; (e) culturing the CAR-T cells in a medium for about four days; and (f) harvesting the CAR-T cells.

In some aspects, the CD4+ and CD8+ T cells are isolated by positive selection. In some aspects, the vector is a virus, a lentivirus, an adenovirus, a retrovirus, an adeno-associated virus (AAV), a transposon, a DNA vector, a mRNA, a lipid nanoparticle (LNP), or a CRISPR-Cas System. In some aspects, the vector is a lentivirus. In some aspects, the lentivirus is added at a multiplicity of invention (MOI) of about 0.25 to about 20. In some aspects, the lentivirus is added at a MOI of about 1 to about 4. In some aspects, the lentivirus is added at a MOI of about 2, or about 4. In some aspects, the cell culture media is increased in volume after step (d). In some aspects, the cell culture media is increased in volume at least 6 fold.

In some aspects, the medium in step (e) is exchanged at least once per day. In some aspects, the medium in step (e) is exchanged every 12 hours. In some aspects, the CAR-T cells or TCR cells are expanded from at least about 1 fold to about 5 fold during step (e). In some aspects, the CAR-T cells or TCR cells are expanded from at least about 1 fold to about 3 fold during step (e). In some aspects, the CAR-T cells or TCR cells are expanded about 2 fold during step (e). In some aspects, the CAR-T cells or TCR cells are expanded about 3 fold during step (e). In some aspects, the CAR binds to STEAP2 or Glypican-3 (GPC3). In some aspects, the CAR encodes an antigen-binding domain that binds to STEAP2 and wherein the antigen-binding domain comprises:
(a) a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6;
(b) a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16;
(c) a VL-CDR1 comprising the amino acid sequence set forth in SEQ ID NO: 21, a VL-CDR2 comprising the amino acid sequence set forth in SEQ ID NO: 22, a VL-CDR3 comprising the amino acid sequence set forth in SEQ ID NO: 23, a VH-CDR1 comprising the amino acid sequence set forth in SEQ ID NO: 24, a VH-CDR2 comprising the amino acid sequence set forth in SEQ ID NO: 25, a VH-CDR3 comprising the amino acid sequence set forth in SEQ ID NO: 26;
(d) a VL-CDR1 comprising the amino acid sequence set forth in SEQ ID NO: 31, a VL-CDR2 comprising the amino acid sequence set forth in SEQ ID NO: 32, a VL-CDR3 comprising the amino acid sequence set forth in SEQ ID NO: 33, a VH-CDR1 comprising the amino acid sequence set forth in SEQ ID NO: 34, a VH-CDR2 comprising the amino acid sequence set forth in SEQ ID NO: 35, a VH-CDR3 comprising the amino acid sequence set forth in SEQ ID NO: 36; or
(e) a VL-CDR1 comprising the amino acid sequence set forth in SEQ ID NO: 41, a VL-CDR2 comprising the amino acid sequence set forth in SEQ ID NO: 42, a VL-CDR3 comprising the amino acid sequence set forth in SEQ ID NO: 43, a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46. In some aspects, the CAR comprises an amino acid sequence having at least about 70%, at least about 75%, at least about 80%, at least about 85%, at least about 90%, at least about 95%, at least about 96%, at least about 97%, at least about 98%, at least about 99%, or 100% sequence identity to the amino acid sequence set forth in SEQ ID NO: 9. In some aspects, the CAR encodes an antigen-binding domain that binds to GPC3 and wherein the antigen-binding domain comprises a heavy chain variable region (VH) and a light chain variable region (VL), wherein the VH comprises a CDR1 comprising the amino acid sequence of SEQ ID NO: 112, a CDR2 comprising the amino acid sequence of SEQ ID NO: 113, and a CDR3 comprising the amino acid sequence of SEQ ID NO: 114, and wherein the VL comprises a CDR1 comprising the amino acid sequence of SEQ ID NO: 115 or SEQ ID NO: 118, a CDR2 comprising the amino acid sequence of SEQ ID NO: 116 or SEQ ID NO: 119, and a CDR3 comprising the amino acid sequence of SEQ ID NO: 117 or SEQ ID NO: 120. In some aspects, the VH comprises the amino acid sequence of SEQ ID NO: 108 or SEQ ID NO: 110, and the VL comprises the amino acid sequence of SEQ ID NO: 109 or SEQ ID NO: 111. In some aspects, the nucleic acid also encodes an armoring molecule.

In some aspects, the armoring molecule comprises a dominant-negative TGFβ receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the armoring molecule comprises the amino acid sequence set forth in SEQ ID NO: 105. In some aspects, the CAR-T cells or TCR cells are formulated in an isotonic solution. In some aspects, the isotonic solution comprises plasmalyte containing human serum albumin. In some aspects, the isotonic solution contains between about 1×10⁶ and about 1×10⁹ CAR-T cells or TCR cells. In some aspects, the isotonic solution contains about 3.4×10⁶ CAR-T cells or TCR cells. In some aspects, the CAR-T cells or TCR cells are a mixture of T_{CM} and T_{SCM} cells. In some aspects, from about 20% to about 50% of the CAR-T cells or TCR cells express CD45RA, CCR7 and CD27, and do not express CD45RO. In some aspects, about 20% to about 30% of the CAR-T cells or TCR cells are T_{SCM} cells and express CD45RA, CCR7 and CD27, and do not express CD45RO. In some aspects, more than 50% of the CAR-T cells or TCR cells express a chimeric antigen receptor or a T-cell receptor. In some aspects, from about 40% to about 60% of the CAR-T Cells or TCR cells express a chimeric antigen receptor or a T-cell receptor. In some aspects, more than 50% of the CAR-T cells or TCR cells express CD8. In some aspects, from about 40% to about 60% of the CAR-T Cells or TCR cells express CD8.

In some aspects, the CAR-T cells or TCR cells have an oxygen consumption rate (OCR) above 100 pmol/min. In some aspects, the CAR-T cells or TCR cells have and OCR from about 50 pmol/min to about 200 pmol/min. In some aspects the CAR-T cells or TCR cells have an extracellular acidification rate (ECAR) above 30 mpH/min. In some aspects, the CAR-T cells or TCR cells have an ECAR about 30 mpH/min to about 60 mpH/min.

### BRIEF DESCRIPTION OF THE DRAWINGS

FIGS. 1A-1D show that GPC3 CAR-T cells expanded in IL-10 or IL-21 alone are less activated than IL-2. FIGS. 1C-1D show the cells are enriched for CAR⁺ and CD8⁺ cells in the presence of IL-21 alone.

FIGS. 2A-2F. FIGS. 2A-2B show cell growth (population doubling times) and cell viability are similar for GPC3 CAR-T cells expanded for 8 days in cell culture medium supplemented with IL-2 only, or IL-21 only, or a combination of IL-2 and IL-21. FIGS. 2C and 2D show the CD4 and CD8 GPC3 CAR-T cells expanded in cell culture medium supplemented with IL-21 only or a combination IL-2 and IL-21 are less differentiated than IL-2 only. FIGS. 2E and 2F show that GPC3 CAR-T cells are able to further expand robustly after day 8 to day 13 when cultured in medium supplemented with IL-2 only or a combination of IL-2 and IL-21.

FIGS. 3A-3F. FIGS. 3A-3B show cell growth (population doubling times) and cell viability are similar for GPC3 CAR-T cells expanded for 8 days in cell culture medium supplemented with IL-2 only, or IL-21 only, or a combination of IL-21 with concentration ranging from 2-10 ng/mL and IL-2 with concentration ranging from 25 to 100 IU/mL. FIG. 3C shows percentages of GPC3 CAR+ cells in CD4 and CD8 T cells are similar between different IL-2 and IL-21 concentrations. FIGS. 3D and 3F show that high IL-2 concentration (>=50 IU/mL) will mask the effect of IL-21 on GPC3 CAR-T cells. FIG. 3F shows that low IL-2 concentration (25 IU/mL) together with IL-21 can enrich CD8 T cells during GPC3 CAR-T cell expansion.

FIGS. 4A-4B show T cell expansion using a 1×10⁹ seed for STEAP2 and GPC3 expressing CAR-T cells.

FIGS. 5A-5B show T cell viability using a 1×10⁹ seed for STEAP2 and GPC3 expressing CAR-T cells.

FIGS. 6A-6B show that the 4 day SMART bioproduction process consistently produce a minimal target of 30% CAR+ cells.

FIGS. 7A-7B show that the 4 day SMART bioproduction process is able to consistently produce a dose of 400×10⁶ CAR+ T cells.

FIGS. 8A-8B shows that the 4-day SMART process produces highly pure CD3+ T cells with a purity greater than 98% in both STEP2 and GPC3 expressing CAR-T cells.

FIGS. 9A-9C show percent STEAP2 CAR expression and TGFβRII armoring expression. FIGS. 9A-9B show high levels of CAR expression and TGFβRII in day 4 processed cells. The day 4 cells showed a linear correlation between CAR and TGFβRII expression. FIG. 9C shows percent GPC3 CAR expression in day 4 processed cells.

FIG. 10 shows the differentiation profile of live CAR+ T cells, and shows that Central Memory (T_{CM}) (CCR7+CD45RO+) is the dominant phenotype for CAR positive T-cells harvested on day-6 whereas CAR-T cells harvested on day-4 show both Stem Cell Memory (T_{SCM}) and T_{CM} phenotypes.

FIGS. 11A-11B show the activation and exhaustion profiles of live CAR+ T cells. CAR+ T cells show increased late-stage activation (CD25+), which activation decreased in day 6 harvested cells compared to day 4 harvested (FIG. 11A). The percentage of cells expressing exhaustion markers is less than 4% for double positive and less than 1% for triple positive for PD1/LAG3/Tim3 (FIG. 11B).

FIGS. 12A-12B show that STEAP2 (FIG. 12A) and GPC3 (FIG. 12B) CAR⁺ T cells demonstrate killing activity on target positive cell lines across a range of E:T ratios.

FIGS. 13A-13B show IFN-γ, TNF-α and IL-2 cytokine release upon target activation by coculturing STEAP2 (FIG. 13A) or GPC3 (FIG. 13B) CAR-T cells with target expressing cells at an E:T Ratio of 1:2. Cytokines released between cells harvested on day 4 or 6 is shown in FIG. 13A.

FIGS. 14A-14E show that SMART GPC3-CAR+ T cells display higher expression of sternness markers and effector function. Further, the SMART cells show lower markers of T-cell exhaustion.

FIGS. 15A-15D shows a schematic of OCR over time. Basal Respiration is the energetic demand of the cell under baseline conditions; Maximal Respiration: respiratory chain operates at maximum capacity→maximum rate of respiration that a cell can achieve; and Spare Respiratory Capacity (SRC): the capability of the cell to respond to an energetic demand or under stress→indicator of cell fitness ΔOCR=OCRₘₐₓ−OCR_{basal}. FIGS. 15A-15D show that SMART CAR-T cells have higher SRC than 12-days TNT CAR-T cells, indicating increased mitochondrial energy reserve, fitness, and adaptability.

FIGS. 16A-16D shows a schematic of ECAR. Glycolysis is the rate of glucose consumption at resting state; glycolytic capacity is the maximum extracellular acidification (ECAR)) rate following shut down of oxidative phosphorylation→cell uses glycolysis to its maximum capacity; and glycolytic reserve is a cell's glycolytic capability to respond to an energetic demand or under stress. FIGS. 16A-16D shows that 4 day SMART CAR-T cells have higher glycolytic reserve than 12-days TNT CAR-T cells, indicating increased capability to perform glycolysis to respond to an energetic demand.

FIGS. 17A-17B show that SMART STEAP2-CAR+ T cells display a greater degree of CAR expression at day 4.

FIGS. 18A-18E show that SMART GPC3-CAR+ T cells have increased antigen-specific secretion of the effector cytokines (FIG. 18A) IFN-γ; (FIG. 18B) IL-2; and (FIG. 18C) IL-21 in a serial kill assay. FIGS. 18D-18E show that SMART GPC3 CAR display enhanced tumor control and increased expansion levels in a serial kill assay as compared to the traditional (TNT) process in two different donors.

FIGS. 19A-19E show GPC-3 SMART CAR⁺ T cells display in vivo dose-dependent tumor control, including TNT and SMART untransduced (UT) controls (FIG. 19B). FIGS. 19C-19D show the IFN-γ profile for 3×10⁶ and 6×10⁶ doses. FIG. 19E shows STEAP2 SMART CAR⁺ T cells display dose-dependent tumor control (0.3 million cells to 6 million cells).

FIGS. 20A-20B shows that CD4/CD8 ratios are retained during cell expansion in prostate cancer (B) compared to a healthy donor (A).

FIGS. 21A-21B show that prostate cancer STEAP2 CAR-T cells are less differentiated as shown by CD62L/CD45RO expression.

FIG. 22 shows in vivo efficacy of administration of SMART CAR-T on tumor volume in a NSG mouse model.

FIG. 23 shows in vivo efficacy of administration of SMART CAR-T compared to traditional 12-day process CAR-T on tumor volume in a NSG MHC class 1/2 knockout mouse model to minimize GvHD.

FIGS. 24A-24D shows 4 day SMART CAR-T have higher SRC than 11 day TNT CAR-T.

FIGS. 25A-25D shows higher concentration of carbonyl cyanide-4 (trifluoromethoxy) phenylhydrazone (FCCP) leads to a greater increase of OCR capacity in 4 day SMART CAR-T cells as compared to 11 day TNT CAR-T cells.

FIGS. 26A-26D show 4 day SMART CAR-T cells had increased glycolysis, glycolytic capacity and glycolytic reserve in comparison to day 11 TNT CAR-T cells.

### DETAILED DESCRIPTION OF THE DISCLOSURE

The present disclosure relates to culturing methods of T cells transduced with chimeric antigen receptors (CARs) that generate a persisting population of T cells that exhibit increased antigen-independent activation.

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a feed medium,” is understood to represent one or more feed mediums. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to ±10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “T cell” or “T lymphocyte” are art-recognized and are intended to include thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, CD4⁻CD8⁻ T cell, or any other subset of T cells. Other illustrative populations of T cells suitable for use in particular aspects include naive T cells and memory T cells.

As used herein, the term “proliferation” refers to an increase in cell division, either symmetric or asymmetric division of cells. In particular aspects, “proliferation” refers to the symmetric or asymmetric division of T cells. “Increased proliferation” occurs when there is an increase in the number of cells in a treated sample compared to cells in a non-treated sample.

The term “expanding” in the method of the invention refers to the process of increasing the number of cells in a cell culture. In the expanding step, cells are fed and culture media is replaced at regular intervals, in one aspect according to a feed regimen. The specific timings and amounts of media added in a particular feed regimen will depend on the cell number and the levels of metabolites in the culture.

As used herein, the term “differentiation” refers to a method of decreasing the potency or proliferation of a cell or moving the cell to a more developmentally restricted state. In particular aspects, differentiated T cells acquire immune effector cell functions.

An “immune effector cell,” is any cell of the immune system that has one or more effector functions (e.g., cytotoxic cell killing activity, secretion of cytokines, induction of ADCC and/or CDC). The illustrative immune effector cells contemplated herein are T lymphocytes, in particular cytotoxic T cells (CTLs; CD8⁺ T cells), TILs, and helper T cells (HTLs; CD4⁺ T cells).

“Modified T cells” refer to T cells that have been modified by the introduction of a polynucleotide encoding an engineered CAR contemplated herein. Modified T cells include both genetic and non-genetic modifications (e.g., episomal or extrachromosomal).

As used herein, the term “genetically engineered” or “genetically modified” refers to the addition of extra genetic material in the form of DNA or RNA into the total genetic material in a cell.

The terms, “genetically modified cells,” “modified cells,” and, “redirected cells,” are used interchangeably.

As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR and/or one or more cytokines. In particular aspects, T cells are modified to express an engineered TCR or CAR without modifying the genome of the cells, e.g., by introducing an episomal vector that expresses the CAR into the cell.

As used herein, a “chimeric antigen receptor (CAR)” means a fused protein comprising an extracellular domain capable of binding to a predetermined antigen, an intracellular segment comprising one or more cytoplasmic domains derived from signal transducing proteins different from the polypeptide from which the extracellular domain is derived, and a transmembrane domain. The “chimeric antigen receptor (CAR)” is sometimes called a “chimeric receptor”, a “T-body”, or a “chimeric immune receptor (CIR).” The phrase “extracellular domain capable of binding to a predetermined antigen” means any proteinaceous molecule or part thereof that can specifically bind to the predetermined antigen. The “intracellular signaling domain” means any oligopeptide or polypeptide domain known to function to transmit a signal causing activation or inhibition of a biological process in a cell, for example, activation of an immune cell such as a T cell. Examples include ILR chain, CD28 and/or CD3ζ.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “ex vivo” refers generally to activities that take place outside an organism, such as experimentation or measurements done in or on living tissue in an artificial environment outside the organism, preferably with minimum alteration of the natural conditions. In particular aspects, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain aspects, such tissues or cells can be collected and frozen, and later thawed for ex vivo treatment. Tissue culture experiments or procedures lasting longer than a few days using living cells or tissue are typically considered to be “in vitro,” though in certain aspects, this term can be used interchangeably with ex vivo.

The term “in vivo” refers generally to activities that take place inside an organism, such as cell self-renewal and expansion of cells. In one aspect, the term “in vivo expansion” refers to the ability of a cell population to increase in number in vivo.

The acronym “SMART” (Shorty-Manipulated Auto-Replicating T-Cells) refers to a T-cell expansion process wherein the cells are cultured in the presence of IL-2 and IL-21.

The acronym “TNT” (Traditionally Nurtured T-Cells) refers to a traditional T-cell expansion process which does not employ IL-21, and typically comprises a cell culture for more than 7 days and/or typically comprises the use of IL-2.

The term “stimulation” refers to a primary response induced by binding of a stimulatory molecule (e.g., a TCR/CD3 complex) with its cognate ligand thereby mediating a signal transduction event including, but not limited to, signal transduction via the TCR/CD3 complex.

A “stimulatory molecule,” refers to a molecule on a T cell that specifically binds with a cognate stimulatory ligand.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands include, but are not limited to CD3 ligands, e.g., an anti-CD3 antibody and CD2 ligands, e.g., anti-CD2 antibody, and peptides, e.g., CMV, HPV, EBV peptides.

The term, “activation” refers to the state of a T cell that has been sufficiently stimulated to induce detectable cellular proliferation. In particular aspects, activation can also be associated with induced cytokine production, and detectable effector functions. The term “activated T cells” refers to, among other things, T cells that are proliferating. Signals generated through the TCR alone are insufficient for full activation of the T cell and one or more secondary or costimulatory signals are also required. Thus, T cell activation comprises a primary stimulation signal through the TCR/CD3 complex and one or more secondary costimulatory signals. Costimulation can be evidenced by proliferation and/or cytokine production by T cells that have received a primary activation signal, such as stimulation through the CD3/TCR complex or through CD2.

A “costimulatory signal,” refers to a signal, which in combination with a primary signal, such as TCR/CD3 ligation, leads to T cell proliferation, cytokine production, and/or upregulation or downregulation of particular molecules (e.g., CD28).

A “costimulatory ligand,” refers to a molecule that binds a costimulatory molecule. A costimulatory ligand may be soluble or provided on a surface. A “costimulatory molecule” refers to the cognate binding partner on a T cell that specifically binds with a costimulatory ligand (e.g., anti-CD28 antibody).

“Autologous,” as used herein, refers to cells from the same subject.

“Allogeneic,” as used herein, refers to cells of the same species that differ genetically to the cell in comparison.

“Syngeneic,” as used herein, refers to cells of a different subject that are genetically identical to the cell in comparison.

“Xenogeneic,” as used herein, refers to cells of a different species to the cell in comparison. In preferred aspects, the cells of the invention are allogeneic.

As used herein, the terms “individual” and “subject” are often used interchangeably and refer to any animal that exhibits a symptom of a cancer that can be treated with the gene therapy vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a cancer, have been diagnosed with a cancer, or are at risk or having a cancer.

By “enhance” or “promote,” or “increase” or “expand” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell death killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a lesser physiological response (i.e., downstream effects) in a cell, as compared to the response caused by either vehicle, a control molecule/composition, or the response in a particular cell lineage. A comparable response is one that is not significantly different or measurable different from the reference response.

### Sources of T Cells

Prior to expansion and genetic modification of the T cells of the invention, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present invention, any number of T cell lines available in the art, may be used. In certain aspects of the present invention, T cells can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Again, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. In some aspects, T cells are isolated by positive selection for CD4 and CD8 expression. For example, in one aspect, T cells are isolated by incubation with anti-CD4/anti-CD8-conjugated beads for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another aspect, the time period is 10 to 24 hours. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD4/CD8 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD4 and/or anti-CD8 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, and HLA-DR. In certain aspects, it may be desirable to enrich for or positively select for regulatory T cells which typically express CD4⁺, CD25⁺, CD62L^{hi}, GITR⁺, and FoxP3⁺. Alternatively, in certain aspects, T regulatory cells are depleted by anti-C25 conjugated beads or other similar method of selection.

For isolation of a desired population of cells by positive or negative selection, the concentration of cells and surface (e.g., particles such as beads) can be varied. In certain aspects, it may be desirable to significantly decrease the volume in which beads and cells are mixed together (i.e., increase the concentration of cells), to ensure maximum contact of cells and beads. For example, in one aspect, a concentration of 2 billion cells/ml is used. In one aspect, a concentration of 1 billion cells/ml is used. In a further aspect, greater than 100 million cells/ml is used. In a further aspect, a concentration of cells of 10, 15, 20, 25, 30, 35, 40, 45, or 50 million cells/ml is used. In yet another aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. In some aspects, the freeze and subsequent thaw step can provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further aspect, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another aspect, the cells are isolated prior to and can be frozen for later use for treatment following B-cell ablative therapy such as agents that react with CD20, e.g., Rituxan.

In a further aspect of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the co-stimulatory signal is bound to a cell surface and the agent providing the primary activation signal is in solution or coupled to a surface. In certain aspects, both agents can be in solution. In another aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof, and both agents are co-immobilized to the same bead in equivalent molecular amounts. In one aspect, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), IL-21, insulin, IFN-7, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). In one aspect, the media is X-VIVO 15 serum-free media containing 1% (v/v) recombinant serum replacement (ITSE-A).

In one aspect, the T cells are cultured in media containing between 10 and 100 IU/mL of recombinant human IL-2. In one aspect, the T cells are cultured in media containing 10, 15, 20, 25, 30, 35, 40, 45, 50, 55, 60, 65, 70, 75, 80, 85, 90, 95, or 100 IU/mL of recombinant human IL-2. In another aspect, the T cells are cultured in media also containing between 0.1 and 0.3 U/mL of recombinant IL-21. In another aspect, the T cells are cultured in media containing IL-2 and 1, 2, 5, 10, 15, 20, 25, 30, 40, 50, 75, or 100 U/mL of recombinant human IL-21. In another aspect, the T cells are culture in media containing IL-2 and 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30 U/mL of recombinant human IL-21. In one aspect, the T cells are cultured in a media containing 40 IU/mL of recombinant human IL-2 and 0.19 U/mL of recombinant human IL-21.

In one aspect of the present invention, the cells cultured for up to 14 days. In another aspect, the mixture may be cultured for 4 days. The T cells can be agitated during any stage of culture. In one aspect, the cells are agitated during cell culture in media containing IL-2 and IL-21. In certain aspects, the T cells harvested on day 4 exhibit higher target independent killing activity compared to CAR-T cells harvested on day 6.

### Chimeric Antigen Receptors (CARs) and T-Cell Receptors (TCRs)

The TCR-engineered T cells express tumor antigen-specific receptors with a and p chains which are produced from high-quality and high-avidity antigen-specific T-cell clones. Separately, CARs are recombinant receptors for antigen, which, in a single molecule, redirect the specificity and function of T lymphocytes and other immune cells. The general premise for their use in cancer immunotherapy is to rapidly generate tumor-targeted T cells, bypassing the barriers and incremental kinetics of active immunization. Once expressed in T cells, the CAR-modified T cells acquire supra-physiological properties that may exert both immediate and long-term effects. The engineering of CARs into T cells requires that T cells be cultured to allow for transduction and expansion. The transduction may utilize a variety of methods, but stable gene transfer is required to enable sustained CAR expression in clonally expanding and persisting T cells. In principle, any cell surface molecule can be targeted through a CAR, thus over-riding tolerance to self-antigens and the antigen recognition gaps in the physiological T cell repertoire that limit the scope of T cell reactivity.

Redirecting immune reactivity towards a chosen antigen is not however the only purpose of smarter CARs, which are designed to accomplish much more than to target and initiate T cell activation. CARs with different strengths and quality of signaling have the potential to modulate T cell expansion and persistence, as well as the strength of T cell activation within the tumor microenvironment, features that dramatically alter the efficacy and safety of tumor-targeted T cells.

Depending on the desired antigen to be targeted, the CAR of the disclosure can be engineered to include the appropriate antigen binding moiety that is specific to the desired antigen target. In one aspect, the CAR specifically recognizes STEAP2 or Glypican-3 (GPC3).

TABLE 1
Sequences.

| SEQ | | |
|---|---|---|
| ID | Description | Sequences |
| 1 | STEAP2 VL | RASQSVNSNLA |
| | CDR1 (40A3) | |
| 2 | STEAP2 VL | GASTRAT |
| | CDR2 | |
| 3 | STEAP2 VL | QQYNNWPFT |
| | CDR3 | |
| 4 | STEAP2 VH | RNSAVWN |
| | CDR1 | |
| 5 | STEAP2 VH | RTYYRSKWYNDYAVSVKS |
| | CDR2 | |
| 6 | STEAP2 | GLLQNNFYYYMDV |
| | VHCDR3 | |
| 7 | STEAP2 VH | QVQLQQSGPGLVKPSQTLSLTCAISGDSVSRNSAVWNWIRQSPSRGLEWLGRTY |
| | | YRSKWYNDYAVSVKSRITINPDTSKNQFSLQVNSVTPEDTAVYYCARGLLQNN |
| | | FYYYMDVWGKGTTVTVSS |
| 8 | STEAP2 VL | EIVMTQSPATLSVSPGERATLSCRASQSVNSNLAWYQQKPGQAPRLLIYGASTR |
| | | ATGIPARFSGSGSGTEFTLTISSLQSEDFAVYYCQQYNNWPFTFGPGTKVDIK |
| 9 | STEAP2 scFv | EIVMTQSPATLSVSPGERATLSCRASQSVNSNLAWYQQKPGQAPRLLIYGASTR |
| | amino acid | ATGIPARFSGSGSGTEFTLTISSLQSEDFAVYYCQQYNNWPFTFGPGTKVDIKGG |
| | sequence | GGSGGGGSGGGGSGGGGSQVQLQQSGPGLVKPSQTLSLTCAISGDSVSRNSAV |
| | | WNWIRQSPSRGLEWLGRTYYRSKWYNDYAVSVKSRITINPDTSKNQFSLQVNS |
| | | VTPEDTAVYYCARGLLQNNFYYYMDVWGKGTTVTVSS |
| 10 | STEAP2 BZ | MLLLVTSLLLCELPHPAFLLIPEIVMTQSPATLSVSPGERATLSCRASQSVNSN |
| | CAR amino acid | LAWYQQKPGQAPRLLIYGASTRATGIPARFSGSGSGTEFTLTISSLQSEDFAVY |
| | sequence | YCQQYNNWPFTFGPGTKVDIKGGGGSGGGGSGGGGSGGGGSQVQLQQSGPGLVK |
| | | PSQTLSLTCAISGDSVSRNSAVWNWIRQSPSRGLEWLGRTYYRSKWYNDYAVSV |
| | | KSRITINPDTSKNQFSLQVNSVTPEDTAVYYCARGLLQNNFYYYMDVWGKGTT |
| | | VTVSSGSESKYGPPCPPCPFWVLVVVGGVLACYSLLVTVAFIIFWVKRGRKKLL |
| | | YIFKQPFMRPVQTTQEEDGCSCRFPEEEEGGCELRVKFSRSADAPAYQQGQNQL |
| | | YNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 11 | STEAP2-3 VL | RASQSVSSNLA |
| | CDR1(40A3GL- | |
| | LO7) | |
| 12 | STEAP2-3 VL | GASTRAT |
| | CDR2 | |
| 13 | STEAP2-3 VL | QQYNNWPFT |
| | CDR3 | |
| 14 | STEAP2-3 VH | RNSAVWN |
| | CDR1 | |
| 15 | STEAP2-3 VH | RTYYRSKWYNDYAVSVKS |
| | CDR2 | |
| 16 | STEAP2-3 VH | GLLQNQFYYYMDV |
| | CDR3 | |
| 17 | STEAP2-3 VH | QVQLQQSGPGLVKPSQTLSLTCAISGDSVSRNSAVWNWIRQSPSRGLEWLGRTY |
| | | YRSKWYNDYAVSVKSRITINPDTSKNQFSLQLNSVTPEDTAVYYCARGLLQNQ |
| | | FYYYMDVWGKGTTVTVSS |
| 18 | STEAP2-3 VL | EIVMTQSPATLSVSPGERATLSCRASQSVSSNLAWYQQKPGQAPRLLIYGASTR |
| | | ATGIPARFSGSGSGTEFTLTISSLQSEDFAVYYCQQYNNWPFTFGPGTKVDIK |
| 21 | STEAP2-2 VL | RSSQSVVHSNGNTYLE |
| | CDR1 | |
| | (mm30D12) | |
| 22 | STEAP2-2 VL | KVSNRFS |
| | CDR2 | |
| 23 | STEAP2-2 VL | FQGSHVPYT |
| | CDR3 | |
| 24 | STEAP2-2 VH | SYGMS |
| | CDR1 | |
| 25 | STEAP2-2 VH | TISSGGSYTFYPDIMKG |
| | CDR2 | |
| 26 | STEAP2-2 VH | RGYGTIYTFSFDS |
| | CDR3 | |
| 27 | STEAP2-2 VH | EVQLVESGGDLVKPGGSLKLSCAASGFSFSSYGMSWVRQTPDKRLEWVATISS |
| | | GGSYTFYPDIMKGRFTISRDNAMNTLYLQMSSLKSEDSAMYYCARRGYGTIYT |
| | | FSFDSWGQGTTLTVSS |
| 28 | STEAP2-2 VL | DVLMTQTPLSLPVSLGDQASISCRSSQSVVHSNGNTYLEWYLQKPGQSPKLLIY |
| | | KVSNRFSGVPDRFSGSGSGTDFTLKISRVEAEDLGVYYCFQGSHVPYTFGGGTK |
| | | LEIK |
| 31 | STEAP2-2 | RSSQSVVHSNANTYLE |
| | Humanized1 VL | |
| | CDR1 | |
| 32 | STEAP2-2 | KVSNRFS |
| | Humanized1 VL | |
| | CDR2 | |
| 33 | STEAP2-2 | FQGSHVPYT |
| | Humanized1 VL | |
| | CDR3 | |
| 34 | STEAP2-2 | SYGMS |
| | Humanized1 VH | |
| | CDR1 | |
| 35 | STEAP2-2 | TISSGGSYTFYPDIMKG |
| | Humanized1 VH | |
| | CDR2 | |
| 36 | STEAP2-2 | RGYGTIYTFSFDA |
| | Humanized1 VH | |
| | CDR3 | |
| 37 | STEAP2-2 | EVQLVESGGGLVKPGGSLRLSCAASGFTFSSYGMSWVRQAPGKRLEWVATISS |
| | Humanized1 | GGSYTFYPDIMKGRFTISRDNSKNTLYLQMNSLRAEDTAVYYCARRGYGTIYTF |
| | VH | SFDAWGQGTTLTVSS |
| 38 | STEAP2-2 | DVVMTQSPLSLPVTLGQPASISCRSSQSVVHSNANTYLEWYLQKPGQSPQLLIY |
| | Humanized1 VL | KVSNRFSGVPDRFSGSGSGTDFTLKISRVEAEDVGVYYCFQGSHVPYTFGQGTK |
| | | LEIK |
| 41 | STEAP2-2 | RSSQSVVHSNANTYLE |
| | Humanized2 VL | |
| | CDR1 | |
| 42 | STEAP2-2 | KVSNRFS |
| | Humanized2 VL | |
| | CDR2 | |
| 43 | STEAP2-2 | FQGSHVPYT |
| | Humanized2 VL | |
| | CDR3 | |
| 44 | STEAP2-2 | SYGMS |
| | Humanized2 VH | |
| | CDR1 | |
| 45 | STEAP2-2 | TISSGGSYTFYPDIMKG |
| | Humanized2 VH | |
| | CDR2 | |
| 46 | STEAP2-2 | RGYGTIYTFSFDA |
| | Humanized2 VH | |
| | CDR3 | |
| 47 | STEAP2-2 | EVQLLESGGGLVQPGGSLRLSCAASGFTFSSYGMSWVRQAPGKRLEWVSTISSG |
| | Humanized2 | GSYTFYPDIMKGRFTISRDNSKNTLYLQMNSLRAEDTAVYYCARRGYGTIYTFS |
| | VH | FDAWGQGTTLTVSS |
| 48 | STEAP2-2 | DVVMTQSPLSLPVTLGQPASISCRSSQSVVHSNANTYLEWYLQKPGQSPQLLIY |
| | Humanized2 VL | KVSNRFSGVPDRFSGSGSGTDFTLKISRVEAEDVGVYYCFQGSHVPYTFGQGTK |
| | | LEIK |
| 57 | STEAP2-4 VH | EVQLVESGGGLVQPGGSLRLSCAASGFTFSSFAMTWVRQAPGKGLEWVSVITY |
| | (40A1) | SGGRTYYADSVKGRFTISRDNSKNTLYLQMNSLRAEDTAVYFCAKDRIAAVGP |
| | | FDYWGQGTLVTVSS |
| 58 | STEAP2-4 VL | DIQLTQSPSFLSASVGDRVTITCRASQGISVYLAWYQQEPGKAPKLLIYAASTLQ |
| | | SGVPSRFSGSGSGTEFTLTISSLQPEDFATYYCQQLNSYPRTFGQGTKVEIK |
| 67 | STEAP2-5 VH | QVQLVQSGAEVKKPGASVKVSCKASGYTFTSYGISWVRQAPGQGLEWMGWIS |
| | (34C1) | GYTGNTNYAQKLQGRVTMTADTSTSTAYMELRSLRSDDTAVYYCARGGSYFD |
| | | YWGQGTLVTVSS |
| 68 | STEAP2-5 VL | DIQMTQSPSTLSASVGDRVTITCRASQSISRWLAWYQQKPGKAPKLLIYKASSLE |
| | | SGVPSRFSGSGSGTEFTLTISSLQPDDFATYYCQQFNSFSPITFGQGTRLEIK |
| 77 | STEAP2-6 VH | QVQLQQPGAELVKPGASVKLSCKASGYTFTSYWMEWVKQRPGQGLEWIGMIH |
| | (6E10) | PNSGITNYNERFKNKATLTVDKSSSTAYMQLSSLTSEDSAVYYCARDHYYILAY |
| | | WGQGTLVTVSA |
| 78 | STEAP2-6 VL | DVLMTQTPLSLPVSLGDQASISCRSSQSVVHSNGNTYLEWYLQKPGQSPKLLIY |
| | | KVSNRFSGVPDRFSGSGSGTDFTLKISRVEAEDLGVYYCFQGSHVPYTFGGGTK |
| | | LEIK |
| 87 | STEAP2-7 VH | QVQLQQPGADLVKPGASVKMSCKASGHTFTNYWVTWVKQRPGQGLEWIGNF |
| | (22F3) | YPGSGIIKYNENFRSKATLTVDISSSTAYMQLSSLTSEDSAVYYCARSKLGDSFY |
| | | FDYWGQGTTLTVSS |
| 88 | STEAP2-7 VL | DVVMTQTPLSLPVSLGNQASISCRSSQSLVHSNGNTYLHWYLQKPGQSPKLLIY |
| 101 | STEAP2 scFv | KVSNRFSGVPDRFSGSGSGTDFTLKISRVEAEDLGVYFCSQSTHVPLTFGAGTKL |
| 102 | nucleic acid | EIK |
| | sequence | GAGATTGTGATGACCCAGAGCCCTGCAACTCTGAGCGTGTCACCCGGAGAA |
| | STEAP2 BZ | AGGGCCACTCTGTCGTGTCGAGCATCGCAGTCCGTGAACTCCAATCTCGCCT |
| | CAR nucleic | GGTACCAGCAGAAGCCTGGGCAGGCCCCGAGGCTGCTCATCTACGGTGCCT |
| | acid sequence | CCACGAGAGCCACGGGAATTCCAGCGCGCTTTAGCGGATCCGGCTCGGGAA |
| | | CCGAGTTCACCCTTACCATCTCATCGCTGCAATCCGAAGATTTCGCCGTGTA |
| | | TTACTGTCAACAGTACAACAACTGGCCGTTCACCTTTGGCCCGGGAACTAAG |
| | | GTCGACATCAAGGGCGGCGGGGGCTCTGGGGGTGGCGGAAGCGGCGGCGG |
| | | CGGATCCGGTGGCGGCGGAAGCCAAGTGCAGCTGCAGCAGTCCGGACCCGG |
| | | ACTCGTGAAGCCGTCCCAGACTCTGTCCCTGACTTGCGCGATTTCCGGCGAT |
| | | TCCGTGTCCCGCAACTCCGCTGTGTGGAACTGGATCCGGCAGTCGCCTTCGA |
| | | GAGGACTGGAGTGGCTGGGACGGACCTACTACCGCTCAAAATGGTATAACG |
| | | ACTATGCTGTGTCCGTCAAGAGCCGCATCACCATTAACCCCGATACCTCCAA |
| | | GAACCAGTTCAGTCTGCAAGTCAACAGCGTGACTCCTGAGGACACCGCCGT |
| | | GTACTACTGCGCCCGGGGTCTGCTGCAAAACAACTTCTACTACTACATGGAC |
| | | GTCTGGGGAAAGGGAACTACTGTGACCGTGTCCTCC |
| | | ATGCTGCTCCTTGTCACATCACTGCTGCTCTGCGAACTGCCCCACCCTGCATT |
| | | CCTCCTGATCCCCGAGATTGTGATGACCCAGAGCCCTGCAACTCTGAGCGTG |
| | | TCACCCGGAGAAAGGGCCACTCTGTCGTGTCGAGCATCGCAGTCCGTGAAC |
| | | TCCAATCTCGCCTGGTACCAGCAGAAGCCTGGGCAGGCCCCGAGGCTGCTC |
| | | ATCTACGGTGCCTCCACGAGAGCCACGGGAATTCCAGCGCGCTTTAGCGGA |
| | | TCCGGCTCGGGAACCGAGTTCACCCTTACCATCTCATCGCTGCAATCCGAAG |
| | | ATTTCGCCGTGTATTACTGTCAACAGTACAACAACTGGCCGTTCACCTTTGG |
| | | CCCGGGAACTAAGGTCGACATCAAGGGCGGCGGGGGCTCTGGGGGTGGCGG |
| | | AAGCGGCGGCGGCGGATCCGGTGGCGGCGGAAGCCAAGTGCAGCTGCAGC |
| | | AGTCCGGACCCGGACTCGTGAAGCCGTCCCAGACTCTGTCCCTGACTTGCGC |
| | | GATTTCCGGCGATTCCGTGTCCCGCAACTCCGCTGTGTGGAACTGGATCCGG |
| | | CAGTCGCCTTCGAGAGGACTGGAGTGGCTGGGACGGACCTACTACCGCTCA |
| | | AAATGGTATAACGACTATGCTGTGTCCGTCAAGAGCCGCATCACCATTAACC |
| | | CCGATACCTCCAAGAACCAGTTCAGTCTGCAAGTCAACAGCGTGACTCCTGA |
| | | GGACACCGCCGTGTACTACTGCGCCCGGGGTCTGCTGCAAAACAACTTCTAC |
| | | TACTACATGGACGTCTGGGGAAAGGGAACTACTGTGACCGTGTCCTCCGGCT |
| | | CCGAATCAAAATACGGTCCGCCATGCCCACCGTGCCCCTTCTGGGTGCTCGT |
| | | GGTCGTCGGAGGGGTTCTGGCCTGCTACTCCCTGCTGGTCACCGTGGCGTTT |
| | | ATCATCTTCTGGGTGAAGCGGGGAAGGAAGAAGCTACTGTACATTTTCAAG |
| | | CAGCCTTTCATGCGGCCTGTGCAGACCACCCAGGAAGAGGACGGCTGTTCCT |
| | | GCCGGTTCCCCGAGGAAGAGGAAGGGGGTTGCGAGCTGCGCGTGAAGTTCA |
| | | GCAGGAGCGCTGATGCCCCAGCGTACCAACAGGGGCAAAACCAGTTGTACA |
| | | ACGAACTGAACCTTGGTCGGCGCGAAGAGTACGACGTGCTTGACAAGCGCC |
| | | GCGGCAGAGATCCCGAGATGGGTGGAAAGCCGCGGCGGAAGAATCCGCAG |
| | | GAAGGGCTCTACAACGAGCTCCAGAAGGACAAGATGGCCGAAGCCTACAGC |
| | | GAAATCGGGATGAAGGGCGAAAGACGCCGGGGAAAAGGACACGACGGACT |
| | | GTACCAGGGGTTGTCGACCGCGACCAAGGACACCTACGACGCCCTGCATAT |
| | | GCAAGCCTTGCCGCCGAGATGA |
| 103 | STEAP2-2 scFv | GATGTTTTGATGACCCAAACTCCTCTCTCCCTGCCTGTCAGTCTTGGAGATCA |
| | nucleic acid | AGCCTCCATCTCTTGCAGATCTAGTCAGAGTGTTGTACATAGTAATGGAAAC |
| | sequence | ACCTATTTAGAATGGTACCTGCAGAAACCAGGCCAGTCTCCAAAGCTCCTGA |
| | | TCTACAAAGTTTCCAACCGATTTTCTGGGGTCCCAGACAGGTTCAGTGGCAG |
| | | TGGATCAGGGACAGATTTCACACTCAAGATCAGCAGAGTGGAGGCTGAGGA |
| | | TCTGGGAGTTTATTACTGCTTTCAAGGTTCACATGTTCCGTACACGTTCGGAG |
| | | GGGGGACCAAGCTGGAAATAAAAGGAGGCGGAGGATCTGGTGGTGGTGGA |
| | | TCTGGCGGCGGAGGAAGTGGTGGCGGAGGCTCTGAGGTGCAGCTGGTGGAG |
| | | TCTGGGGGAGACTTAGTGAAGCCTGGAGGGTCCCTGAAACTCTCCTGTGCCG |
| | | CCTCTGGATTCTCTTTCTCCTCTTATGGCATGTCTTGGGTTCGCCAGACTCCA |
| | | GACAAGAGGCTGGAATGGGTCGCAACCATTAGTAGTGGTGGTAGTTACACC |
| | | TTCTATCCCGACATTATGAAGGGGCGATTCACCATCTCCAGAGACAATGCCA |
| | | TGAACACCCTGTACCTGCAAATGAGCAGTCTGAAGTCTGAGGACTCAGCCA |
| | | TGTATTACTGTGCAAGACGGGGCTACGGTACTATCTACACGTTTTCCTTTGA |
| | | CTCCTGGGGCCAAGGCACCACTCTCACAGTCTCCAGC |
| 104 | Dominant- | ATGGGACGCGGGCTGCTTCGAGGACTCTGGCCACTTCATATCGTGTTGTGGA |
| | negative TGFβ | CTCGCATCGCTTCAACCATTCCGCCGCACGTGCAGAAGTCCGTGAACAATGA |
| | receptor type 2 | CATGATCGTGACCGACAACAACGGTGCAGTGAAGTTCCCACAGCTGTGCAA |
| | nucleic acid | GTTCTGCGATGTCAGATTCAGCACTTGCGACAACCAGAAGTCCTGCATGTCA |
| | sequence | AACTGCTCCATCACCTCCATCTGCGAGAAGCCTCAAGAGGTCTGCGTGGCCG |
| | | TGTGGCGGAAGAACGACGAGAACATCACCCTGGAAACCGTGTGCCACGATC |
| | | CGAAGCTGCCTTATCACGACTTCATTCTGGAAGATGCCGCCTCGCCCAAGTG |
| | | TATCATGAAAGAAAAGAAAAAGCCCGGAGAAACGTTCTTCATGTGCTCGTG |
| | | TAGCTCCGACGAGTGCAACGACAACATTATCTTTAGCGAAGAGTACAACAC |
| | | TTCCAACCCTGACCTCCTGCTCGTGATTTTTCAAGTCACCGGCATTTCCCTGC |
| | | TGCCCCCGCTGGGCGTGGCGATCTCGGTGATCATTATCTTCTACTGTTACCG |
| | | GGTCAATAGGCAG |
| 105 | Dominant- | MGRGLLRGLWPLHIVLWTRIASTIPPHVQKSVNNDMIVTDNNGAVKFPQLCKF |
| | negative TGFβ | CDVRFSTCDNQKSCMSNCSITSICEKPQEVCVAVWRKNDENITLETVCHDPKLP |
| | receptor type 2 | YHDFILEDAASPKCIMKEKKKPGETFFMCSCSSDECNDNIIFSEEYNTSNPDLLLV |
| | amino acid | IFQVTGISLLPPLGVAISVIIIFYCYRVNRQ |
| | sequence | |
| 106 | STEAP2 BZ | MLLLVTSLLLCELPHPAFLLIPEIVMTQSPATLSVSPGERATLSCRASQSVNSNLA |
| | CAR-T2A- | WYQQKPGQAPRLLIYGASTRATGIPARFSGSGSGTEFTLTISSLQSEDFAVYYCQ |
| | Dominant- | QYNNWPFTFGPGTKVDIKGGGGSGGGGSGGGGSGGGGSQVQLQQSGPGLVKP |
| | negative TGFβ | SQTLSLTCAISGDSVSRNSAVWNWIRQSPSRGLEWLGRTYYRSKWYNDYAVSV |
| | receptor type 2 | KSRITINPDTSKNQFSLQVNSVTPEDTAVYYCARGLLQNNFYYYMDVWGKGTT |
| | amino acid | VTVSSGSESKYGPPCPPCPFWVLVVVGGVLACYSLLVTVAFIIFWVKRGRKKLL |
| | sequence | YIFKQPFMRPVQTTQEEDGCSCRFPEEEEGGCELRVKFSRSADAPAYQQGQNQL |
| | | YNELNLGRREEYDVLDKRRGRDPEMGGKPRRKNPQEGLYNELQKDKMAEAYS |
| | | EIGMKGERRRGKGHDGLYQGLSTATKDTYDALHMQALPPRGSGEGRGSLLTC |
| | | GDVEENPGPMGRGLLRGLWPLHIVLWTRIASTIPPHVQKSVNNDMIVTDNNGA |
| | | VKFPQLCKFCDVRFSTCDNQKSCMSNCSITSICEKPQEVCVAVWRKNDENITLE |
| | | TVCHDPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQ |
| 107 | STEAP2 BZ | ATGCTGCTCCTTGTCACATCACTGCTGCTCTGCGAACTGCCCCACCCTGCATT |
| | CAR-T2A- | CCTCCTGATCCCCGAGATTGTGATGACCCAGAGCCCTGCAACTCTGAGCGTG |
| | Dominant- | TCACCCGGAGAAAGGGCCACTCTGTCGTGTCGAGCATCGCAGTCCGTGAAC |
| | negative TGFβ | TCCAATCTCGCCTGGTACCAGCAGAAGCCTGGGCAGGCCCCGAGGCTGCTC |
| | receptor type 2 | ATCTACGGTGCCTCCACGAGAGCCACGGGAATTCCAGCGCGCTTTAGCGGA |
| | nucleic acid | TCCGGCTCGGGAACCGAGTTCACCCTTACCATCTCATCGCTGCAATCCGAAG |
| | sequence | ATTTCGCCGTGTATTACTGTCAACAGTACAACAACTGGCCGTTCACCTTTGG |
| | | CCCGGGAACTAAGGTCGACATCAAGGGCGGCGGGGGCTCTGGGGGTGGCGG |
| | | AAGCGGCGGCGGCGGATCCGGTGGCGGCGGAAGCCAAGTGCAGCTGCAGC |
| | | AGTCCGGACCCGGACTCGTGAAGCCGTCCCAGACTCTGTCCCTGACTTGCGC |
| | | GATTTCCGGCGATTCCGTGTCCCGCAACTCCGCTGTGTGGAACTGGATCCGG |
| | | CAGTCGCCTTCGAGAGGACTGGAGTGGCTGGGACGGACCTACTACCGCTCA |
| | | AAATGGTATAACGACTATGCTGTGTCCGTCAAGAGCCGCATCACCATTAACC |
| | | CCGATACCTCCAAGAACCAGTTCAGTCTGCAAGTCAACAGCGTGACTCCTGA |
| | | GGACACCGCCGTGTACTACTGCGCCCGGGGTCTGCTGCAAAACAACTTCTAC |
| | | TACTACATGGACGTCTGGGGAAAGGGAACTACTGTGACCGTGTCCTCCGGCT |
| | | CCGAATCAAAATACGGTCCGCCATGCCCACCGTGCCCCTTCTGGGTGCTCGT |
| | | GGTCGTCGGAGGGGTTCTGGCCTGCTACTCCCTGCTGGTCACCGTGGCGTTT |
| | | ATCATCTTCTGGGTGAAGCGGGGAAGGAAGAAGCTACTGTACATTTTCAAG |
| | | CAGCCTTTCATGCGGCCTGTGCAGACCACCCAGGAAGAGGACGGCTGTTCCT |
| | | GCCGGTTCCCCGAGGAAGAGGAAGGGGGTTGCGAGCTGCGCGTGAAGTTCA |
| | | GCAGGAGCGCTGATGCCCCAGCGTACCAACAGGGGCAAAACCAGTTGTACA |
| | | ACGAACTGAACCTTGGTCGGCGCGAAGAGTACGACGTGCTTGACAAGCGCC |
| | | GCGGCAGAGATCCCGAGATGGGTGGAAAGCCGCGGCGGAAGAATCCGCAG |
| | | GAAGGGCTCTACAACGAGCTCCAGAAGGACAAGATGGCCGAAGCCTACAGC |
| | | GAAATCGGGATGAAGGGCGAAAGACGCCGGGGAAAAGGACACGACGGACT |
| | | GTACCAGGGGTTGTCGACCGCGACCAAGGACACCTACGACGCCCTGCATAT |
| | | GCAAGCCTTGCCGCCGAGAGGATCCGGAGAGGGGAGGGGAAGCCTCCTCAC |
| | | TTGCGGCGATGTGGAGGAAAACCCGGGTCCTATGGGACGCGGGCTGCTTCG |
| | | AGGACTCTGGCCACTTCATATCGTGTTGTGGACTCGCATCGCTTCAACCATT |
| | | CCGCCGCACGTGCAGAAGTCCGTGAACAATGACATGATCGTGACCGACAAC |
| | | AACGGTGCAGTGAAGTTCCCACAGCTGTGCAAGTTCTGCGATGTCAGATTCA |
| | | GCACTTGCGACAACCAGAAGTCCTGCATGTCAAACTGCTCCATCACCTCCAT |
| | | CTGCGAGAAGCCTCAAGAGGTCTGCGTGGCCGTGTGGCGGAAGAACGACGA |
| | | GAACATCACCCTGGAAACCGTGTGCCACGATCCGAAGCTGCCTTATCACGA |
| | | CTTCATTCTGGAAGATGCCGCCTCGCCCAAGTGTATCATGAAAGAAAAGAA |
| | | AAAGCCCGGAGAAACGTTCTTCATGTGCTCGTGTAGCTCCGACGAGTGCAA |
| | | CGACAACATTATCTTTAGCGAAGAGTACAACACTTCCAACCCTGACCTCCTG |
| | | CTCGTGATTTTTCAAGTCACCGGCATTTCCCTGCTGCCCCCGCTGGGCGTGG |
| | | CGATCTCGGTGATCATTATCTTCTACTGTTACCGGGTCAATAGGCAG |
| 108 | GPC3 VH | EVQLLESGGGLVQPGGSLRLSCAASGFTFSSYAMSWVRQAPGKGLEWVSAISG |
| | | SGGSTYYADSVKGRFTISRDNSKNTLYLQMNSLRAEDTAVYYCARGKRYFDY |
| | | WGQGTMVTVSS |
| 109 | GPC3 VL | SYELTQPPSASGTPGQRVTISCSGGSSNIGSNTVNWFRQLPGTAPKLLVYFNNQR |
| | | PSGVPDRFSGSKSGTSASLAIGGLQSDDEADYYCVAWDDSLNAPVFGGGTKVT |
| | | VL |
| 110 | GPC3-2 VH | EVQLLESGGGLVQPGGSLRLSCAASGFTFSSYAMSWVRQAPGKGLEWVSAISG |
| | | SGGSTYYADSVKGRFTISRDNSKNTLYLQMNSLRAEDTAVYYCAKGKRYFDY |
| | | WGQGTMVTVSS |
| 111 | GPC3-2 VL | QSVLTQPPSASGTPGQRVTISCSGGSSDIGSNTVNWYQQLPGTAPKLLIYYNNQR |
| | | PSGVPDRFSGSKSGTSASLAISGLQSEDEADYYCATWDDRMYSPVFGGGTKLTV |
| | | L |
| 112 | GPC3 and | GFTFSSYAMS |
| | GPC3-2 VH | |
| | CDR1 | |
| 113 | GPC3 and | AISGSGGSTYYADSVKG |
| | GPC3-2 VH | |
| | CDR2 | |
| 114 | GPC3 and | GKRYFDY |
| | GPC3-2 VH | |
| | CDR3 | |
| 115 | GPC3 VL CDR1 | SGGSSNIGSNTVN |
| 116 | GPC3 VL CDR2 | FNNQRPS |
| 117 | GPC3 VL CDR3 | VAWDDSLNAPV |
| 118 | GPC3-2 VL | SGGSSDIGSNTVN |
| | CDR1 | |
| 119 | GPC3-2 VL | YNNQRPS |
| | CDR2 | |
| 120 | GPC3-2 VL | ATWDDRMYSPV |
| | CDR3 | |

### Armoring Molecule

Disclosed herein are polynucleotides comprising (a) a nucleotide sequence encoding a CAR, wherein the CAR comprises an antigen-binding domain, and (b) a nucleotide sequence encoding an armoring molecule. One approach to making CAR-T cells that are more resistant to tumor-associated immunosuppression is called “armoring.” Armoring is the molecular manipulation of a CAR-T cell to express one or more “armoring molecules” that can counter immunosuppression. For example, investigators reported modifying CAR-T cells to secrete PD-1-blocking single-chain variable fragments (scFv), which improved CAR-T cell anti-tumor activity in mouse models of PD-L1+ hematologic and solid tumors (Rafiq, S., Yeku, O., Jackson, H. et al. Targeted delivery of a PD-1-blocking scFv by CAR-T cells enhances anti-tumor efficacy in vivo. Nat Biotechnol 36, 847-856 (2018)). Others studies have demonstrated the effectiveness of armoring T cells with a dominant-negative TGFβ receptor type 2 (TGFβRIIDN) armoring molecule to neutralize the suppressive effects of TGFβ on T cells (Bollard et al., Tumor-Specific T-Cells Engineered to Overcome Tumor Immune Evasion Induce Clinical Responses in Patients With Relapsed Hodgkin Lymphoma, J Clin Oncol 36(11):1128-1139 (2018)). Currently, at least one clinical study is investigating the effectiveness of armoring anti-PSMA-CAR-T cells with a TGFβRIIDN armoring molecule for treating castrate-resistant prostate cancer (NCT03089203).

In some aspects, the armoring molecule comprises a dominant-negative TGFβ receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the armoring molecule comprises the amino acid sequence set forth in SEQ ID NO: 105.

Metabolic Testing of CAR-T cells or TCR cells

In some aspects, the metabolic activity of CAR-T or TCR cells were measured using a Seahorse® assay. The Seahorse® assay measures the extracellular flux of OCR and ECAR. OCR reflects the rate at which cells consume oxygen during oxidative phosphorylation, a process that occurs in the mitochondria. ECAR measures the production of protons resulting from glycolysis, the metabolic pathway that generates energy from glucose. In some aspects, CAR-T cells or TCR cells are added to specialized microplates with wells that contain sensors for detecting OCR and ECAR changes. The cells are exposed to experimental conditions, such as different concentrations of drugs or metabolic substrates, and the OCR and ECAR are measured at intervals. In some aspects, the Seahorse® assay is performed using 0.5 μM FCCP. In some aspects the Seahorse® assay is performed using 2 μM FCCP. In some aspects, the CAR-T cells or TCR cells have an OCR above 100 pmol/min. In some aspects, the CAR-T cells or TCR cells have an OCR above 40 pmol/min. In some aspects, the CAR-T cells or TCR cells have an OCR above 150 pmol/min. In some aspects, the CAR-T cells or TCR cells have an OCR from about 50 pmol/min to about 200 pmol/min. In some aspects, the CAR-T cells or TCR cells have an ECAR above 30 mpH/min. In some aspects, the CAR-T cells or TCR cells have an ECAR above 50 mpH/min. In some aspects, the CAR-T cells or TCR cells have an ECAR above 30 mpH/min. In some aspects, the CAR-T cells or TCR cells have an ECAR from about 30 mpH/min to about 60 mpH/min.

### Examples

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### Example 1: IL-2 and IL-21

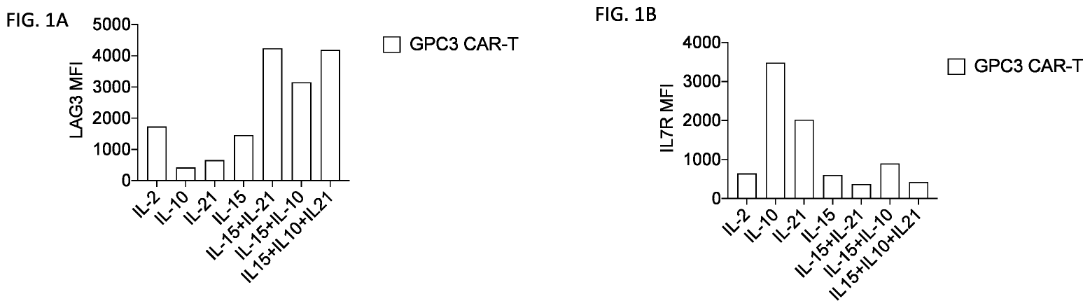

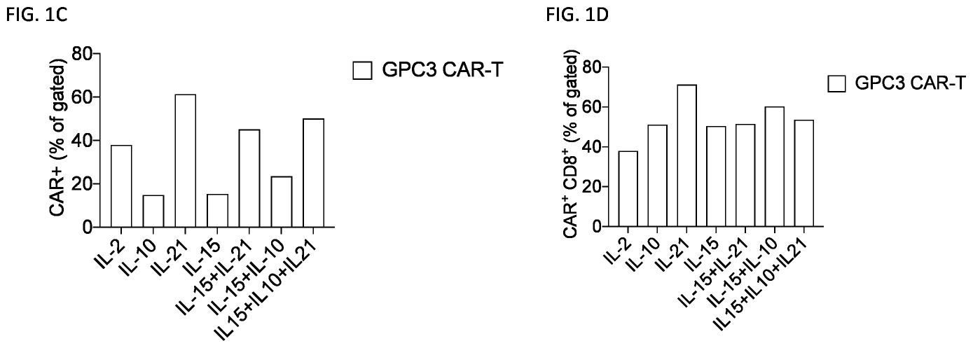

Expansion in the presence of IL-21 results in less differentiated cells and higher proportion of CAR+CD8+ cells. Purified human T cells were seeded in AIM-V medium containing 5% human serum, 1% penicillin-streptomycin (Invitrogen), and 1% antibiotic-antimycotic (Invitrogen) at a concentration of 0.2E6 cells/mL+interleukin (IL)-2 (300 IU/mL) (Peprotech). T cells were activated with anti-CD3/CD28 Dynabeads (Invitrogen) according to the manufacturer's protocol. 24 hours later, lentivirus was added to the wells, and plates were centrifuged at 2000 g, 37° C., for 2 hours. After centrifugation, cells were washed and resuspended in fresh media containing IL-2 (300 IU/mL), IL-21 (10 ng/mL, R&D Systems), IL-10 (10 ng/mL, R&D Systems), and IL-15 (10 ng/mL, R&D Systems) as indicated. Plates were placed in a 37° C., 5% CO2 incubator and cells were split as necessary to maintain cell density at ˜1E6 cells/mL. After 10 days cells were harvested and analyzed by flow cytometry (FIG. 1A-D).

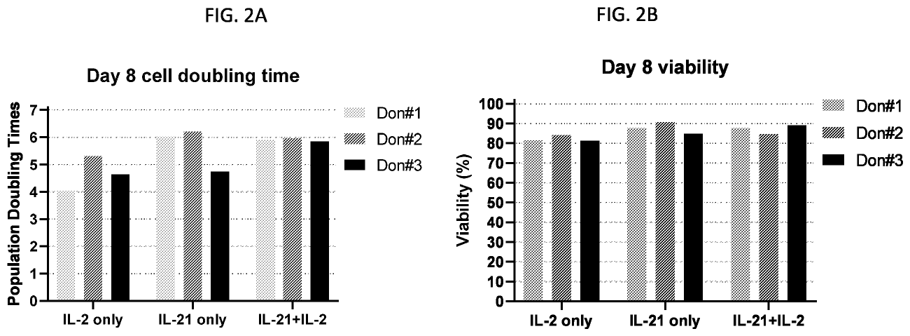

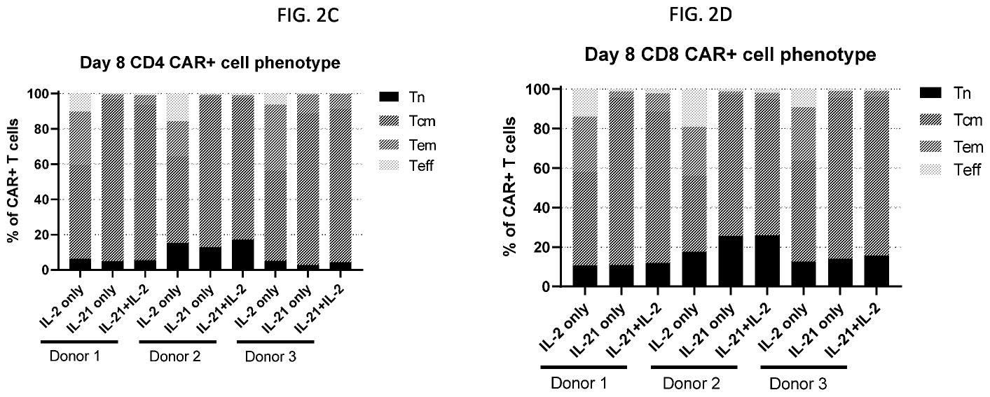

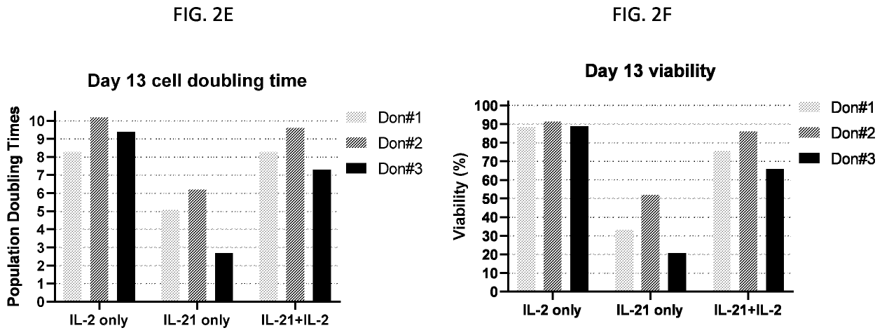

The combination of IL-2 and IL-21 results in better phenotype and long-term cell expansion in TNT cells harvested at day 8. Selected total T cells (CD4 and CD8) were seeded in X-VIVO 15 medium (Lonza) supplemented with 5% CTS Serum Replacement (Thermo Fisher) at 1.5E6 viable cells/mL in 125 mL shake flask at 10% working volume agitated at 51 rpm on day 0. ImmunoCult CD3/CD28/CD2 T cell activator (Stemcell Technologies) was added to cell culture at 25 μL/mL to activate T cells immediately after seeding. After two days of culture in incubator at 37° C. and 5% CO₂, GPC3 LVV was added to cell culture at MOI of 10, and agitation rate was increased to 169 rpm to enhance LVV transduction. On day 3, 0.9E6 viable cells were transferred and cultured in 100 mL of X-VIVO 15 medium+5% (v/v) CTS serum replacement supplemented with IL-2 (Akron) only at 100 IU/mL, or IL-21 (Akron) at 10 ng/mL alone, or 25 IU/mL IL-2 and 10 ng/mL IL-21. On day 6, a second dose of IL-2 at the same concentration as on day 3 was added to each well without mixing. On day 8, 5E6 viable cells were passaged and cultured in 100 mL of the same medium with the fresh cytokines at the same concentrations. On day 10, 2^{nd} dose of IL-2 was added to cell culture. On day 8 and day 13, cells were harvested for cell counting and expression of CD3, CD4, CD8, GPC3 CAR, CD45RO, CD45RA, CD62L, and CCR7 were analyzed by flow cytometry (FIGS. 2A-2F).

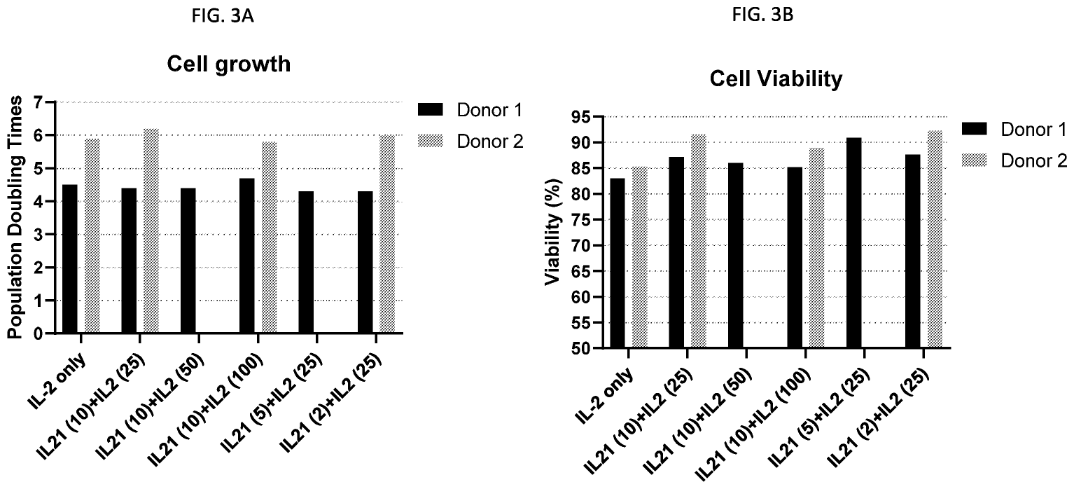

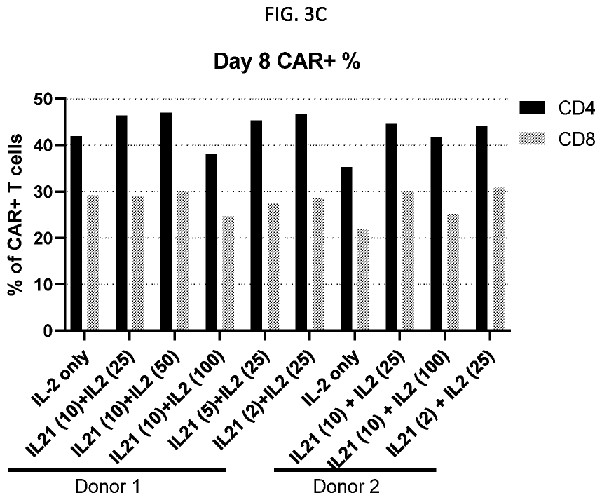

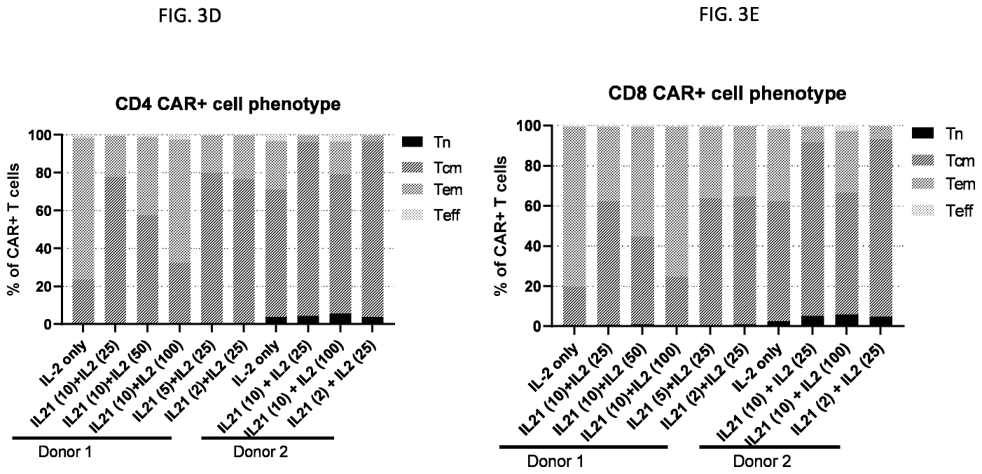

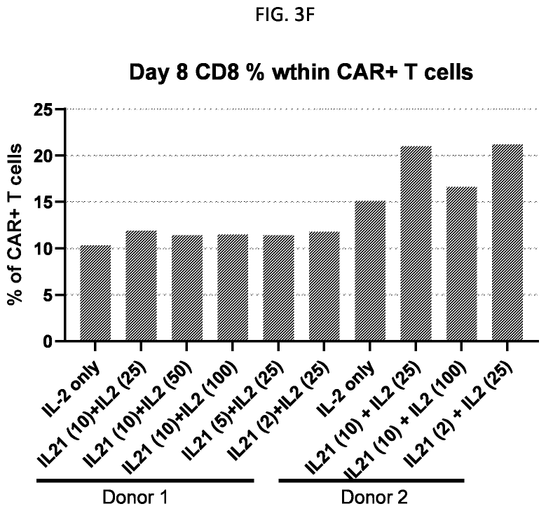

High IL-2 concentration can mask the effect of IL-21. Selected total T cells (CD4 and CD8) were seeded in X-VIVO 15 medium (Lonza) supplemented with 5% CTS Serum Replacement (Thermo Fisher) at 1.5E6 viable cells/mL in 125 mL shake flask at 10% working volume agitated at 51 rpm on day 0. ImmunoCult CD3/CD28/CD2 T cell activator (Stemcell Technologies) was added to cell culture at 25 μL/mL to activate T cells immediately after seeding. After two days of culture in incubator at 37° C. and 5% CO2, GPC3 LVV was added to cell culture at MOI of 10, and agitation rate was increased to 169 rpm to enhance LVV transduction. On day 3, 0.9E6 viable cells were transferred and cultured in 100 mL of X-VIVO 15 medium+5% (v/v) CTS serum replacement supplemented with IL-2 (Akron) only at 100 IU/mL, or 25 IU/mL IL-2 and 10 ng/mL IL-21, or 50 IU/mL IL-2 and 10 ng/mL IL-21, or 100 IU/mL IL-2 and 10 ng/mL IL-21, or 25 IU/mL IL-2 and 5 ng/mL IL-21, or 25 IU/mL IL-2 and 2 ng/mL IL-21. On day 6, a second dose of IL-2 at the same concentration as on day 3 was added to each well without mixing. On day 8, cells were harvested for cell counting and expression of CD3, CD4, CD8, GPC3 CAR, CD45RO, CD45RA, CD62L, and CCR7 were analyzed by flow cytometry (LSR Fortessa from BD Biosciences) (FIGS. 3A-3F).

### Example 2: SMART 4 Day CAR-T Cell Culture Process

Apheresis Biological Starting Material (BSM) from patients was received from the clinical site within pre-defined window of collection, shipped at 2-8° C. The BSM was washed on the Cytiva Sefia S2000 to remove majority of RBC and platelets using Flexcell program, and then formulated in 1:1 of PlasmaLyte A (Baxter) with 5% (w/v) human serum albumin (HSA): CryoStor® CS10, split into two 70 mL/CS250 bags (OriGen), and cryopreserved using a controlled rate freezer prior to storage into vapor phase of LN₂.

Day 0: When manufacturing starts, frozen half leukopak was thawed under controlled condition using PlasmaTherm (Plasma Therm), and CD4 plus CD8 T-lymphocytes were isolated using GMP anti-CD4 and anti-CD8 CliniMACS microbeads (Miltenyi) on the Miltenyi Prodigy®. After isolation, 1.0E+09 purified CD3 T-cells were added to the cultivation chamber of Miltenyi Prodigy® tubing set and activated with Miltenyi T cell TransAct™ via CD3/28 at v/v=1:17.5 on the same day, and cultured overnight in 70 mL of complete X-VIVO 15 serum-free media (Lonza) containing 1% (v/v) recombinant serum replacement (ITSE-A), 40 IU/mL recombinant human IL-2 and 0.19 U/mL recombinant human IL-21.

Day 1: The following day, cells were transduced with lentiviral vector at a predefined multiplicity of infection. After two hours of lentivirus addition, fresh cell culture medium was added to bring cell culture volume to 250 mL.

Day 2 to day 4: cells were continued to culture and expand on days 2, 3, 4. 180 mL of cell culture medium was exchanged with 180 mL fresh complete media containing 1% (v/v) recombinant serum replacement (ITSE-A), 40 IU/mL recombinant human IL-2 and 0.19 U/mL recombinant human IL-21 every 12 hours.

Day 4: the cells were washed with harvest buffer (PlasmaLyte A (Baxter) with 5% (w/v) human serum albumin (HSA)) and concentrated by volume reduction to produce Drug Substance (DS). Samples were taken for analysis.

### Example 3: SMART CAR-T Cell Culture Process (Shake Flask Scale Down Model)

Day 0: For scale down model studies, CD4 plus CD8 T-lymphocytes were enriched from frozen biological starting materials on Prodigy or manually using GMP anti-CD4 and anti-CD8 CliniMACS microbeads (Miltenyi). After isolation, 1.0×10⁸ purified CD3 T-cells were added to 125 mL shake flask and activated with Miltenyi T cell TransAct™ via CD3/28 at v/v=1:17.5 on the same day, and cultured overnight in 7 mL of complete X-VIVO 15 serum-free media (Lonza) containing 1% (v/v) recombinant serum replacement (ITSE-A), 40 IU/mL recombinant human IL-2 and 0.19 U/mL recombinant human IL-21. The shake flask was placed on orbital shaker at 50 rpm.

Day 1: The following day, cells were transduced with lentiviral vector at a predefined multiplicity of infection. After two hours of lentivirus addition, fresh cell culture medium was added to bring cell culture volume to 25 mL. After volume increase, the agitation rate of orbital shaker was increased to 65 rpm.

Day 2: Cell culture was split into two equal fractions (˜12 mL each) and 5 mL of spent medium is removed from cell culture. Each cell culture fractions in 125 mL shake flask was added with 18 mL (total 25 mL) of complete X-VIVO 15 serum-free media (Lonza) containing 1% (v/v) recombinant serum replacement (ITSE-A), 40 IU/mL recombinant human IL-2 and 0.19 U/mL recombinant human IL-21.

Day 3: cell culture was exchanged with 18 mL fresh complete media containing 1% (v/v) recombinant serum replacement (ITSE-A), 40 IU/mL recombinant human IL-2 and 0.19 U/mL recombinant human IL-21 every 24 hours.

On day 4, the cells were washed with harvest buffer (PlasmaLyte A (Baxter) with 5% (w/v) human serum albumin (HSA)) and concentrated by volume reduction to produce Drug Substance (DS). Samples were taken for analysis.

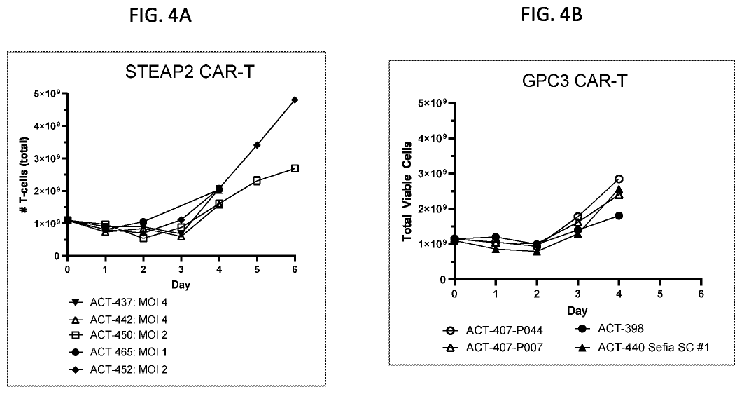

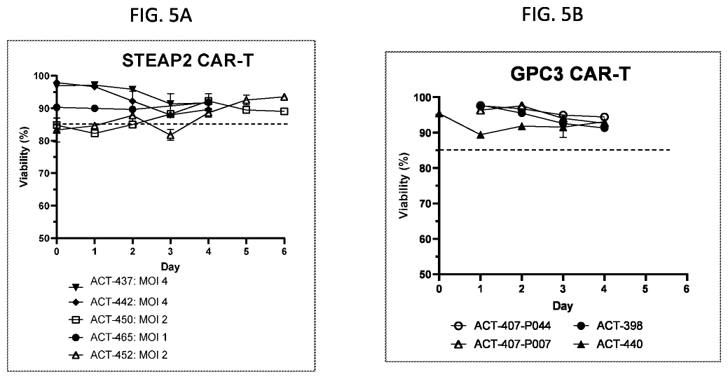

Using the shaker flask process, excellent T cell expansion was seen for seedings of 1×10⁹ cells for both STEAP2 and GPC3 CAR-T cells. Total viable cell number was determined, and the expanded T cells also maintain high viability (FIGS. 4A-4B and FIGS. 5A-5B). CAR-T cells that are expanded in IL-10 or IL-21 were shown to be less activated, and further, CAR-T cells were enriched for CAR⁺ and CD8⁺ cells in the presence of IL-21 alone.

### Example 4: Analytical Testing of SMART 4-Day Process Versus Traditional Culture Process (TNT)

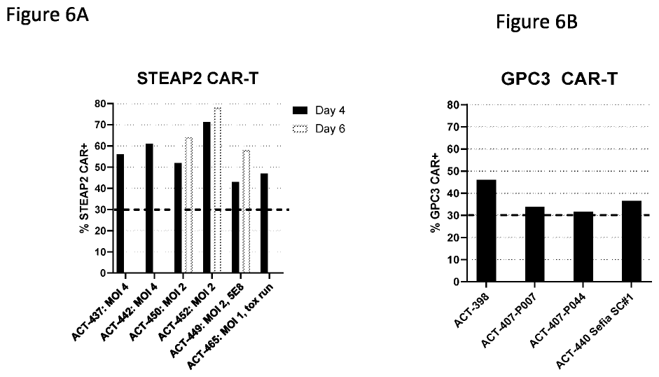

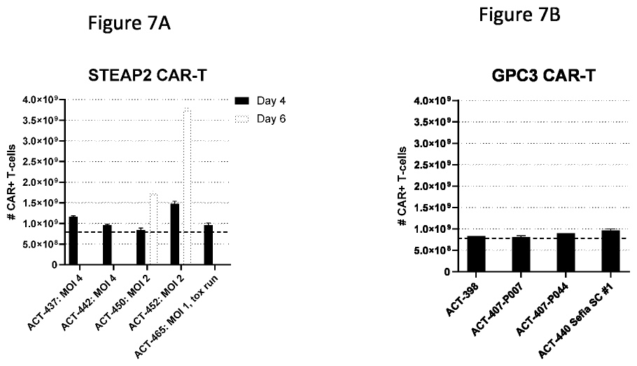

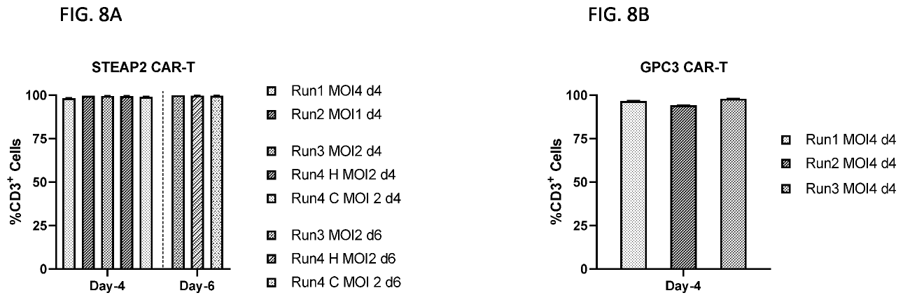

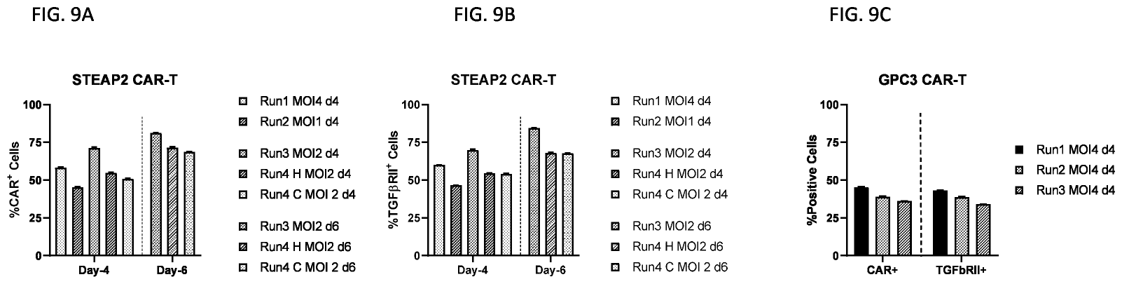

The relative purity of SMART process T cells was evaluated. As shown in FIGS. 8A-8B, the T cell population was highly pure population of cells with an overall CD3 positivity of at least 98% for both STEAP2 and GPC3 CAR-T cells. The level of CAR expression (FIGS. 6A-6B) showed a correlation between CAR and TGFβRII expression in STEAP2 (FIGS. 9A-9B) and GPC3 (FIG. 9B) CAR-T cells. Similarly high expression levels of STEAP2 CAR were visible when starting with either prostate cancer patient derived PBMCs (FIG. 9A, Run4 C) and Healthy donor PBMCs (FIG. 9A, Run4 H). Interestingly, for STEAP2 CAR expression, the percentage of CAR+ T cells further increased when harvesting the cells on day 6 compared to day 4 (FIG. 9A).

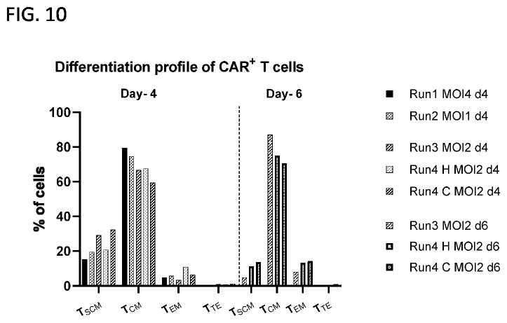

The differentiation profile of live CAR+ T cells showed a dominant early memory phenotype (FIG. 10). As shown in FIG. 10, central memory (T_{CM}) (CCR7+CD45RO+) is the dominant phenotype for CAR positive T-cells harvested on day-6 whereas CAR-T cells harvested on day-4 shows both Stem Cell Memory (T_{SCM}) and T_{CM}. Differentiation to T_{CM} cells increases with increasing the duration of expansion to 6-days. Importantly, the phenotypes of CAR positive T cells from cancer patient and healthy donors were comparable.

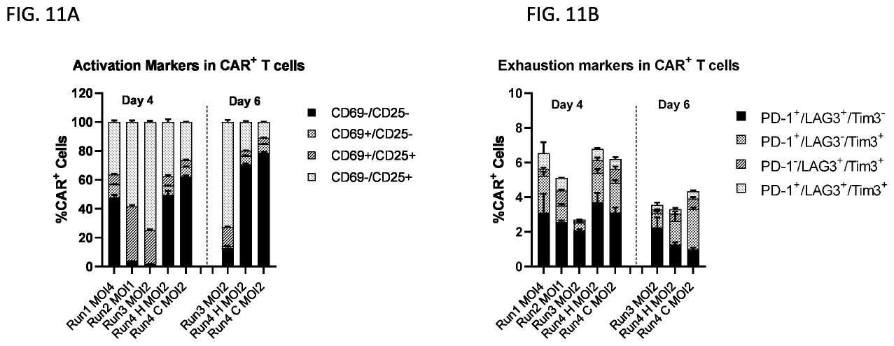

Live CAR+ T Cells also showed more late-stage activation profile with less than 1% of cells are PD1/LAG3/TIM3 triple positive (FIGS. 11A-11B). In the activation profile, CAR+ T cells showed more late-stage activation (CD25+). Activation is slightly reduced in CAR-T cells when harvested on day 6 compared to cells harvested on day 4. In the exhaustion profile, overall, the percentage of cells expressing exhaustion markers was very low, less than 4% for double positive and less than 1% for triple positive for PD1/LAG3/Tim3. Slight differences were observed in the expression of exhaustion markers for CAR-T cells generated from 4-days vs 6-days processed cells.

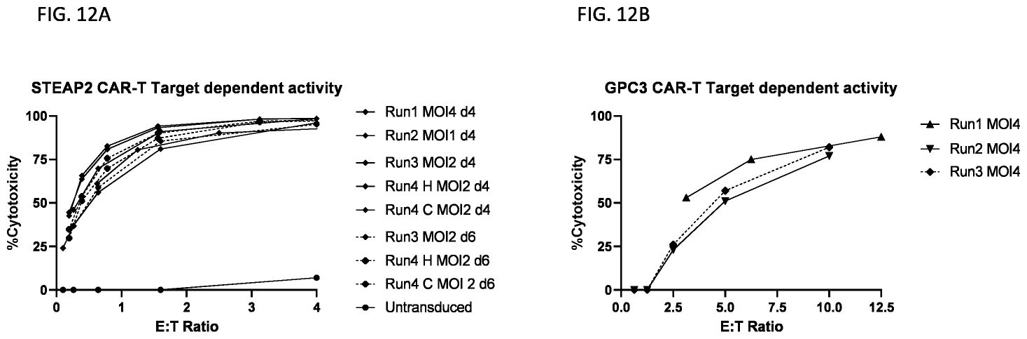

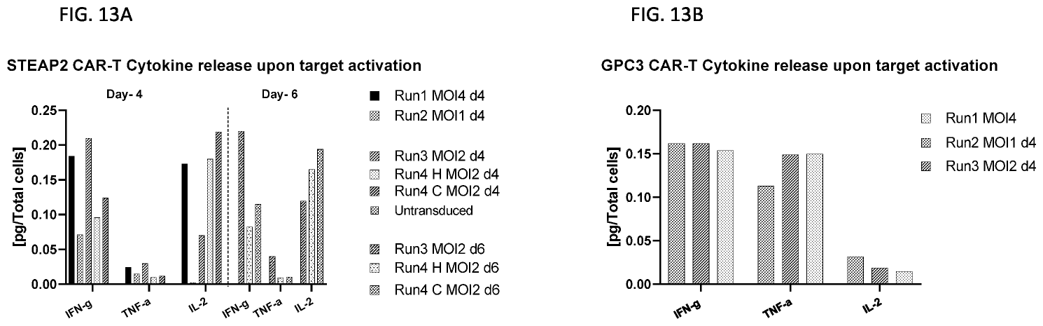

Functionality of STEAP2 and GPC3 CAR-T cells is shown in FIGS. 12A-12B and 11A-11B. As shown in FIGS. 12A-12B, STEAP2 and GPC3 CAR-T cells demonstrate target dependent killing activity across a range of E:T ratios. Cytokine release is observed when cells are co-cultured at an E:T Ratio of 1:2 with target expressing cell lines as shown in FIGS. 13A-13B for STEAP2 and GPC3 CAR-T cells.

### Example 5: Biological Characterization of SMART CAR-T Cells

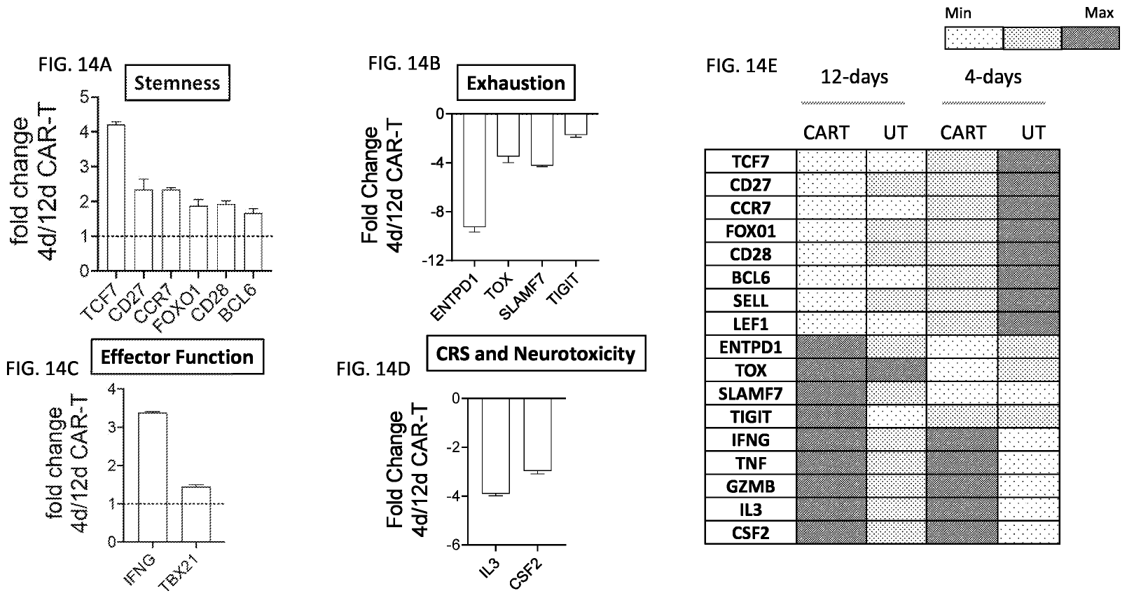

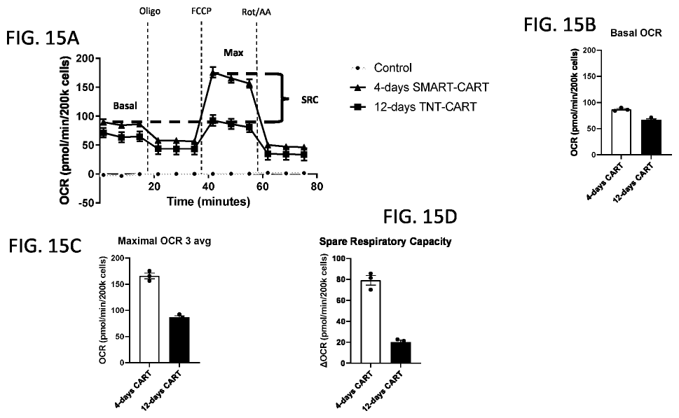

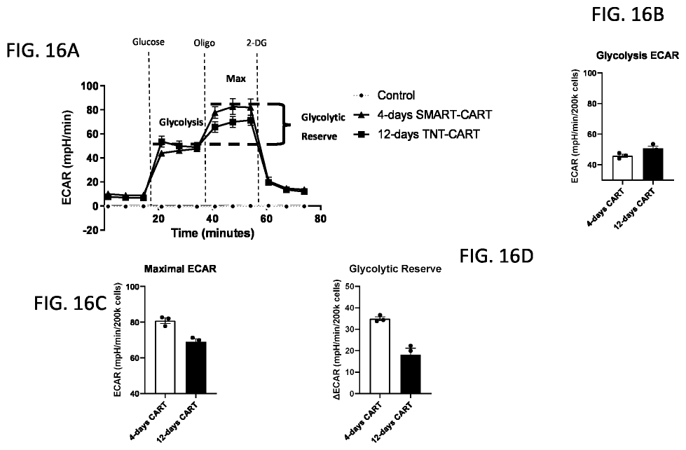

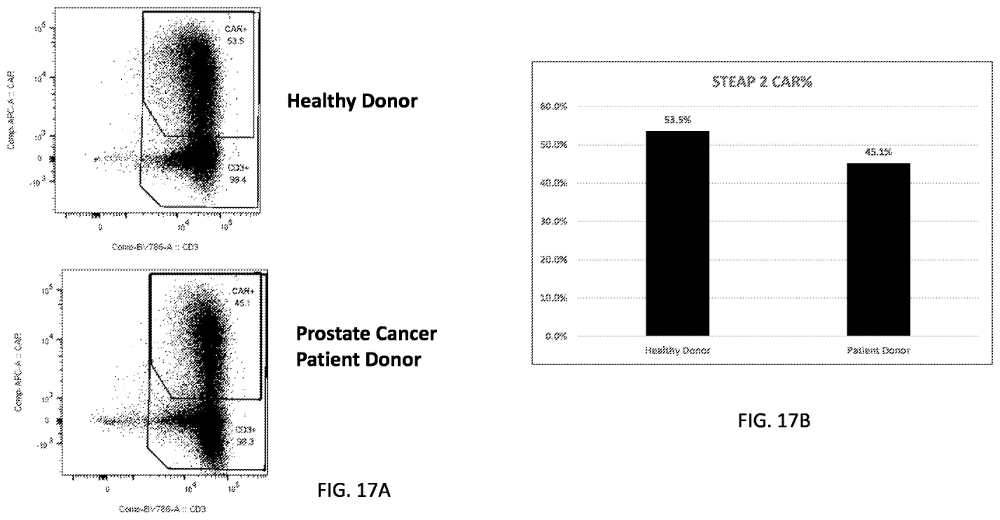

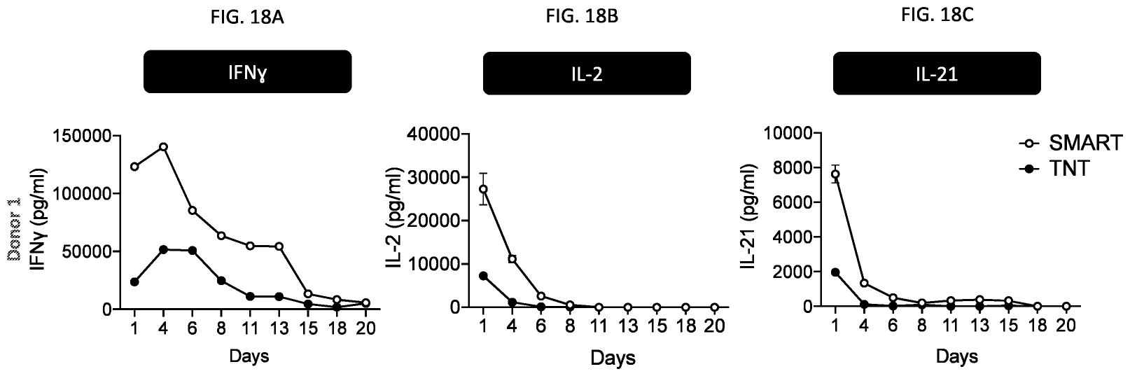

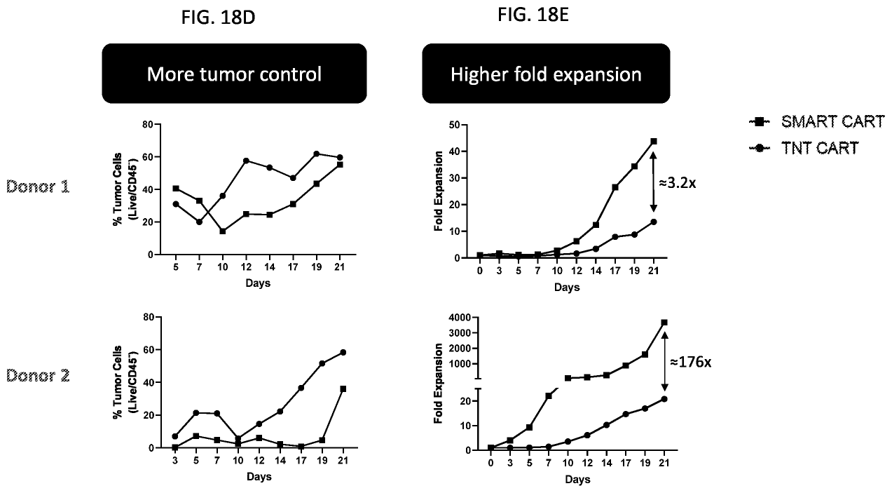

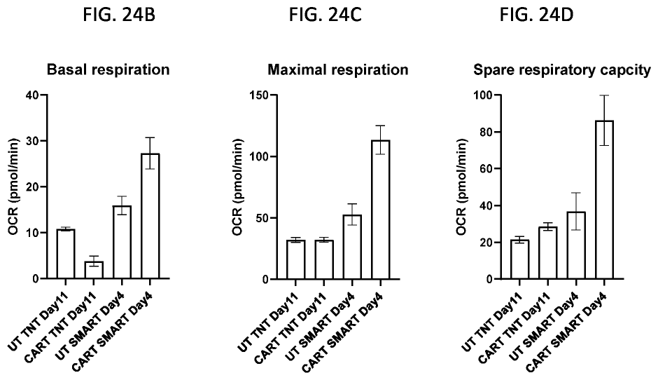

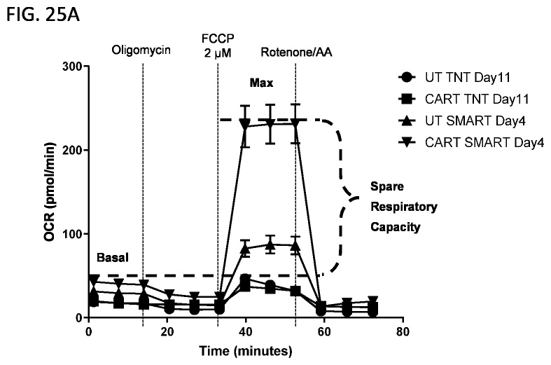

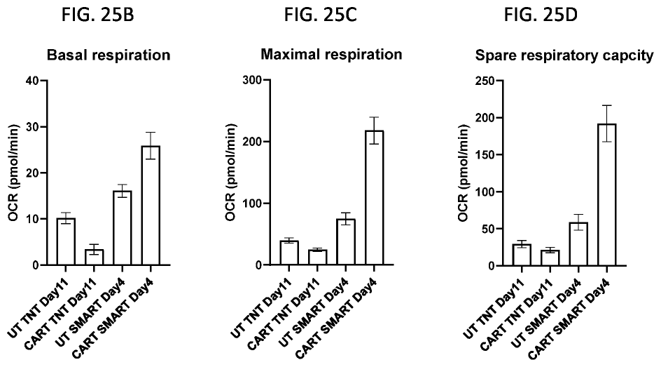

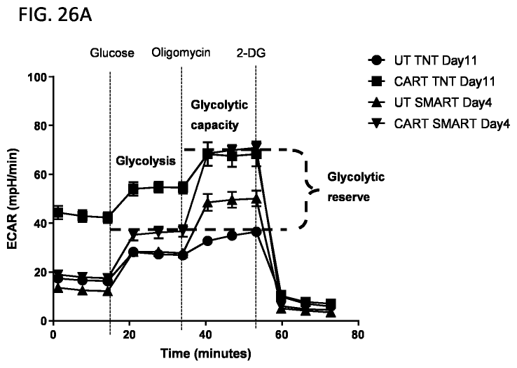

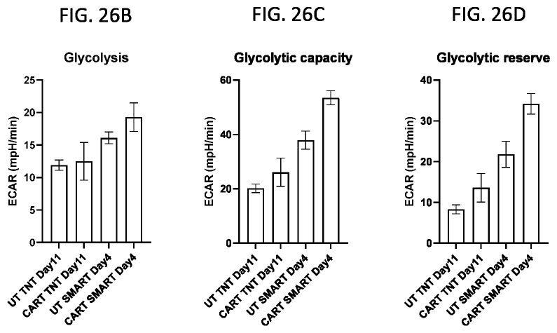

Both GPC3 and STEAP2 SMART CAR-T cells were analyzed to determine the mechanism for their increased activity relative to CAR-T cells produced from traditional processes. It was hypothesized that the shorter SMART expansion process produces cells with higher stemness and fitness. Expression of stemness genes from 4 day and 12 day processes were analyzed for TCF7, CD27, CCR7, FOXO1, CD28, and BCL6. As shown in FIGS. 14A-14E, 15A-15D, and 16A-16D, 4 day T cells showed higher expression of stemness genes and high metabolic fitness as compared to TNT CAR-T cells. This translated into both better expression of the STEAP2 CAR by day 4 (FIGS. 17A-17B) and higher fold expansion of SMART 4 day GPC3 CAR-T cells versus a traditional TNT process in an in vitro serial kill assay (FIGS. 18D-18E). SMART CAR-T cells also had increased antigen-specific secretion of effector cytokines. As shown in FIGS. 18A-18C, SMART CAR-T cells produced higher levels of IFNγ, IL-2, and IL-21 in a serial kill assay. Metabolic fitness showed 4 day SMART CAR-T cells have higher OCR (FIGS. 24A-24D, 25A-25D) and ECAR than 11 day TNT CAR-T (FIGS. 26A-26D).

### Example 6: In Vivo Efficacy of SMART CAR-T Cells

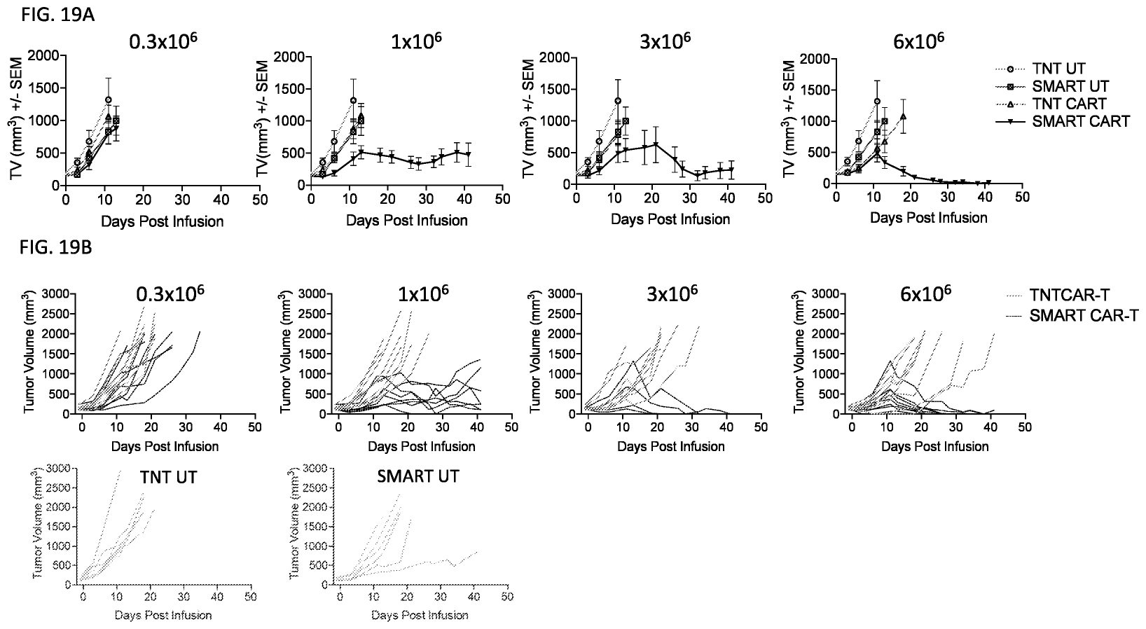

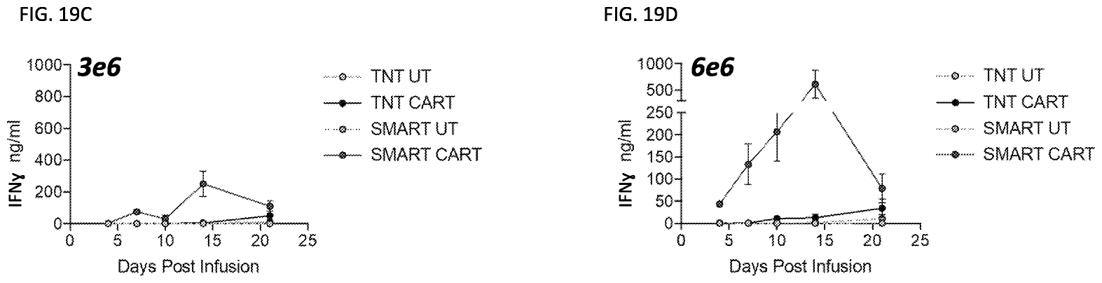

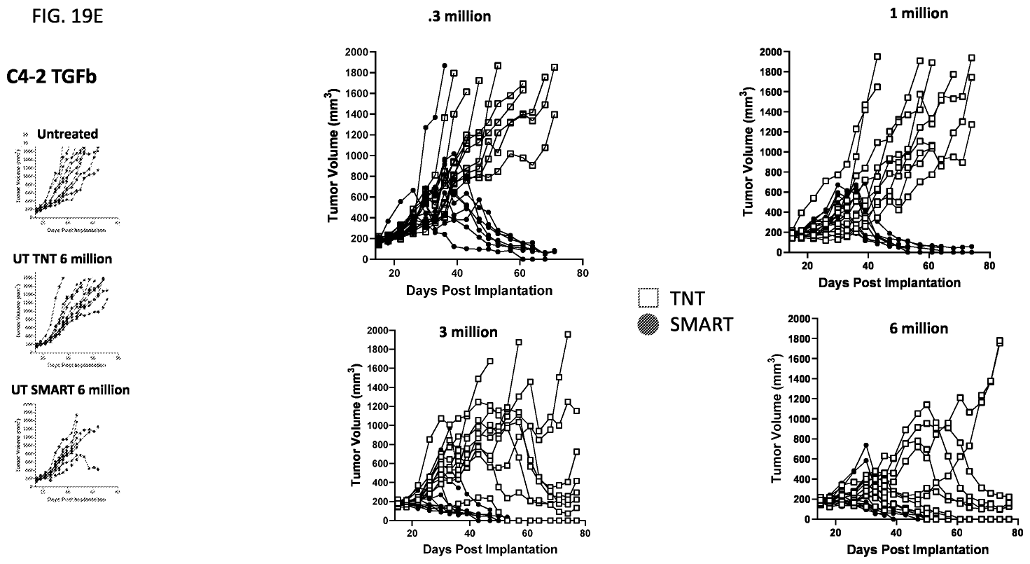

To determine the impact of the TNT and SMART cell in vitro phenotypes on their activity in an in vivo setting, NSG mice were implanted with GPC3 positive HUH7 tumors overexpressing human TGFβ. When tumors reached an average size of 200 mm³, mice were randomized and IV dosed with the doses indicated in FIGS. 19A-19B of TNT or SMART GPC3 CAR-T cells. Tumor volumes and body weight were monitored twice per week throughout the study to reveal superior tumor control with the SMART CAR+ T cells compared to the TNT CAR+ T cells at all doses tested (FIGS. 19A-19B). Mice were bled at the indicated days and IFNγ in the serum was analyzed by MSD (FIGS. 19C-19D). Significant reduction in tumor volume was seen following administration of SMART CAR-T cells, and it correlated with higher concentration of IFNγ in the serum.

Separately, the same experiment was performed using STEAP2 CAR-T cells using implanted STEAP2 positive C4-2 tumors that were exogenously expressing human TGFβ. When tumors reached an average size of 175 mm³, mice were randomized and IV dosed with a titration range of TNT or SMART STEAP2 CAR-T cells, as indicated in FIG. 19E. Tumor volumes and body weight were monitored twice per week throughout the study to reveal superior tumor control with the SMART CAR+ T cells compared to the TNT CAR+ T cells at all doses tested.

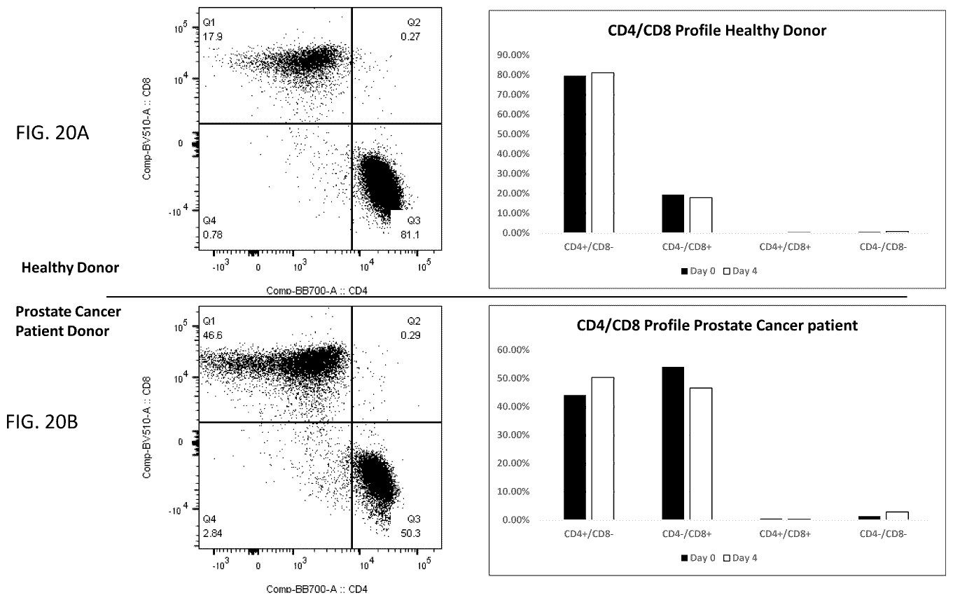

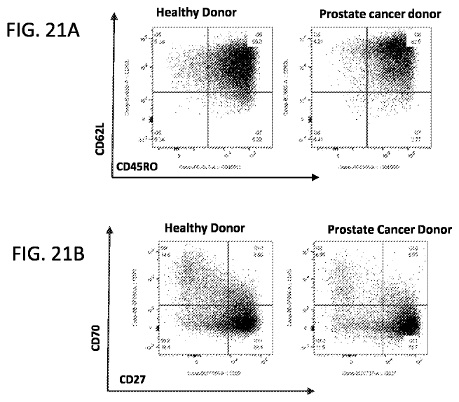

The CD4/CD8 ratio from prostate cancer T cells that underwent expansion were also analyzed. As shown in FIGS. 20A-20B, T cells were double stained and analyzed by FACS. These prostate cancer T cells were also less differentiated than cells from a healthy donor as shown by CD62L/CD45RO expression. (FIGS. 21A-21B).

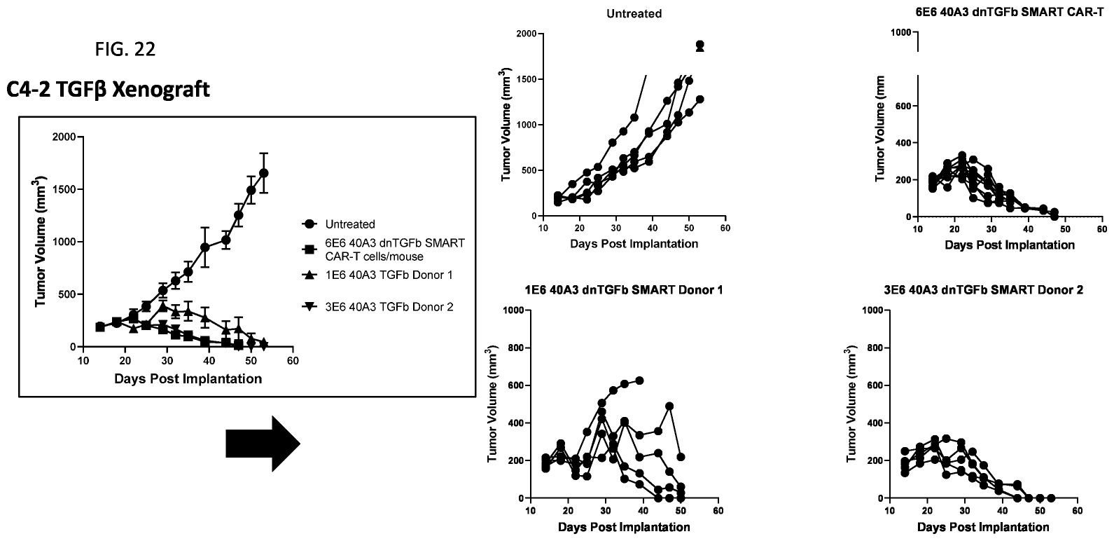

STEAP2 positive C4-2 cells overexpressing exogenous human TGFb were implanted into male NSG mice. Upon tumor size reaching an average of 175 mm³ mice were randomized into treatment groups and dosed with various amounts of SMART CAR-T cells from two different donors, as indicated in FIG. 22. Significant reduction in tumor volume was seen following administration of SMART CAR-T cells from both donors.

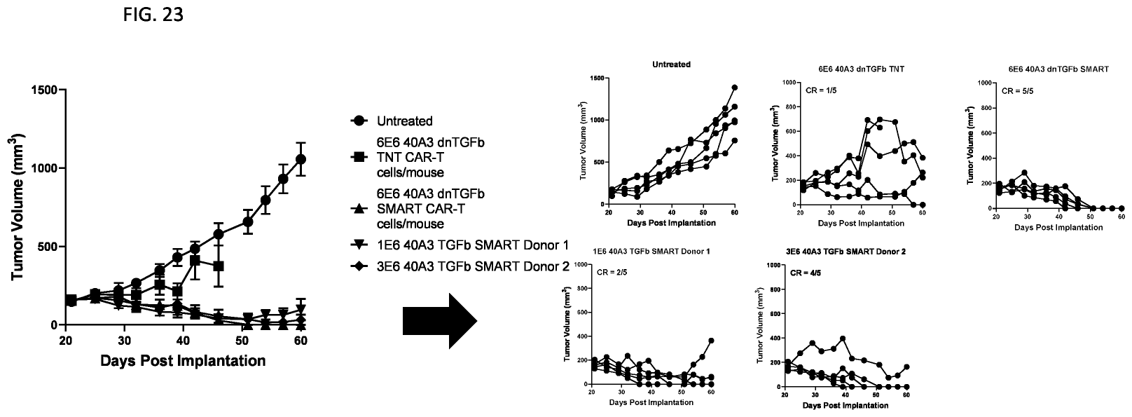

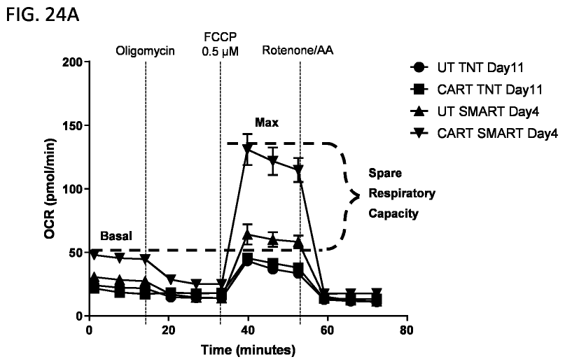

A similar experiment was run in which the C4-2 TGFb cells were implanted into NSG MHC class 1/class 2 knockout mice to assuage potential contributions of GvHD. This study compared the 6e6 dose of TNT and SMART 40A3 CAR-T cells from the same donor. This comparison revealed superior tumor growth inhibition and more overall complete responders in the SMART CAR-T treated group. The second donor SMART material at 1e6 and 3e6 was also efficacious in this setting leading to ⅖ and ⅘ complete responders, respectively. The same degree of tumor volume reduction was not seen for 12-day (TNT) CAR-T cells (FIG. 23).

1. A method of expanding a population of T cells comprising: (a) isolating CD3⁺ T cells from a sample; (b) culturing the CD3⁺ T cells in a culture media that comprises human interleukin 21 (IL-21); (c) activating the CD3⁺ T cells; (d) transducing the CD3⁺ T cells with a vector comprising a nucleic acid encoding a chimeric antigen receptor (CAR) or a T-Cell Receptor (TCR) to produce CAR-T cells or T-cell Receptor (TCR) cells; (e) culturing the CAR-T cells in a medium; and (f) harvesting the CAR-T cells or T-cell Receptor (TCR) cells.

2. A method of manufacturing a T cell therapeutic comprising: (a) obtaining a sample comprising a population of CD3⁺ T cells; (b) culturing the CD3⁺ T cells in a culture media that comprises human interleukin 21 (IL-21); (c) activating the CD3⁺ T cells; (d) transducing the CD3⁺ T cells with a vector comprising a nucleic acid encoding a chimeric antigen receptor (CAR) or a T-Cell Receptor (TCR) to produce CAR-T cells or T-cell Receptor (TCR) cells; (e) culturing the CAR-T cells or T-cell Receptor (TCR) cells in a medium; and (f) harvesting the CAR-T cells or T-cell Receptor (TCR) cells.

3. A method of expanding a population of T cells comprising: (a) isolating CD4⁺ and CD8⁺ T cells from a sample to form a population of CD3⁺ T cells; (b) culturing the CD3⁺ T cells in a culture media containing human interleukin 21 (IL-21); (c) activating the CD3⁺ T cells; (d) transducing the CD3⁺ T cells with a vector comprising a nucleic acid encoding a chimeric antigen receptor (CAR) or a T-Cell Receptor (TCR) to produce CAR-T cells or T-cell Receptor (TCR) cells; (e) culturing the CAR-T cells or T-cell Receptor (TCR) cells in a medium; and (f) harvesting the CAR-T cells or T-cell Receptor (TCR) cells.

4. The method of claim 1, wherein the culture media further comprises human interleukin 2 (IL-2).

5. The method of claim 1, wherein (d) comprises transducing the CD3⁺ T cells with a vector comprising a nucleic acid encoding a CAR to produce CAR-T cells.

6. (canceled)

7. The method of claim 1, wherein from about 1×10⁶ to about 1×10⁹ CD3⁺ T cells are cultured in the culture media in (b).

8. (canceled)

9. The method of claim 7, wherein the CD3⁺ T cells in (c) are cultured for about one day or about two days.

10-11. (canceled)

12. The method of claim 9, wherein the CD3+ T cells in (c) are activated with an anti-CD3 antibody or CD3-binding fragment thereof, and an anti-CD28 antibody or a CD28-binding fragment thereof.

13-14. (canceled)

15. The method of claim 12, wherein the CAR-T cells or TCR cells are cultured in (e) from about four to about six days.

16-17. (canceled)

18. The method of claim 15, wherein the concentration of human IL-21 is from about 0.01 U/mL to about 0.3 U/mL, and the concentration of human IL-2 is from about 5 IU/mL to about 100 IU/mL.

19-24. (canceled)

25. The method of claim 18, wherein the vector is a lentivirus and wherein the lentivirus is added at a multiplicity of infection (MOI) of about 0.25 to about 20.

26-32. (canceled)

33. The method of claim 25, wherein the CAR-T cells or TCR cells are expanded from at least about 1 fold to about 5 fold during (e).

34-37. (canceled)

38. The method of claim 1, wherein the CAR encodes an antigen-binding domain that binds to STEAP2 and wherein the antigen-binding domain comprises:
(a) a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6;
(b) a VL-CDR1 comprising the amino acid sequence set forth in SEQ ID NO: 11, a VL-CDR2 comprising the amino acid sequence set forth in SEQ ID NO: 12, a VL-CDR3 comprising the amino acid sequence set forth in SEQ ID NO: 13, a VH-CDR1 comprising the amino acid sequence set forth in SEQ ID NO: 14, a VH-CDR2 comprising the amino acid sequence set forth in SEQ ID NO: 15, a VH-CDR3 comprising the amino acid sequence set forth in SEQ ID NO: 16;
(c) a VL-CDR1 comprising the amino acid sequence set forth in SEQ ID NO: 21, a VL-CDR2 comprising the amino acid sequence set forth in SEQ ID NO: 22, a VL-CDR3 comprising the amino acid sequence set forth in SEQ ID NO: 23, a VH-CDR1 comprising the amino acid sequence set forth in SEQ ID NO: 24, a VH-CDR2 comprising the amino acid sequence set forth in SEQ ID NO: 25, a VH-CDR3 comprising the amino acid sequence set forth in SEQ ID NO: 26;
(d) a VL-CDR1 comprising the amino acid sequence set forth in SEQ ID NO: 31, a VL-CDR2 comprising the amino acid sequence set forth in SEQ ID NO: 32, a VL-CDR3 comprising the amino acid sequence set forth in SEQ ID NO: 33, a VH-CDR1 comprising the amino acid sequence set forth in SEQ ID NO: 34, a VH-CDR2 comprising the amino acid sequence set forth in SEQ ID NO: 35, a VH-CDR3 comprising the amino acid sequence set forth in SEQ ID NO: 36; or
(e) a VL-CDR1 comprising the amino acid sequence set forth in SEQ ID NO: 41, a VL-CDR2 comprising the amino acid sequence set forth in SEQ ID NO: 42, a VL-CDR3 comprising the amino acid sequence set forth in SEQ ID NO: 43, a VH-CDR1 comprising the amino acid sequence set forth in SEQ ID NO: 44, a VH-CDR2 comprising the amino acid sequence set forth in SEQ ID NO: 45, a VH-CDR3 comprising the amino acid sequence set forth in SEQ ID NO: 46.

39. (canceled)

40. The method of claim 1, wherein the CAR encodes an antigen-binding domain that binds to GPC3 and wherein the antigen-binding domain comprises a heavy chain variable region (VH) and a light chain variable region (VL), wherein the VH comprises a CDR1 comprising the amino acid sequence of SEQ ID NO: 112, a CDR2 comprising the amino acid sequence of SEQ ID NO: 113, and a CDR3 comprising the amino acid sequence of SEQ ID NO: 114, and wherein the VL comprises a CDR1 comprising the amino acid sequence of SEQ ID NO: 115 or SEQ ID NO: 118, a CDR2 comprising the amino acid sequence of SEQ ID NO: 116 or SEQ ID NO: 119, and a CDR3 comprising the amino acid sequence of SEQ ID NO: 117 or SEQ ID NO: 120.

41. (canceled)

42. The method of claim 1, wherein the nucleic acid also encodes an armoring molecule and wherein the armoring molecule comprises a dominant-negative TGFβ receptor type 2 (TGFβRIIDN).

43-49. (canceled)

50. The method of claim 1, wherein the CAR-T cells or TCR cells are a mixture of T_{CM} and T_{SCM} cells and wherein from about 15% to about 50% of the CAR-T cells or TCR cells are T_{SCM} cells and said T_{SCM} cells express CD45RA, CCR7, and CD27, and do not express CD45RO.

51-52. (canceled)

53. The method of claim 1, wherein more than 50% of the CAR-T cells or TCR cells express a chimeric antigen receptor or a T-cell receptor.

54. (canceled)

55. The method of claim 1, wherein more than 50% of the CAR-T cells or TCR cells express CD8.

56. (canceled)

57. The method of claim 1, wherein the CAR-T cells or TCR cells have an oxygen consumption rate (OCR) above 100 pmol/min.

58. (canceled)

59. The method of claim 1, wherein the CAR-T cells or TCR cells have an extracellular acidification rate (ECAR) above 30 mpH/min.

60. (canceled)
